(19) United States
(12) Patent Application Publication
(10) Pub. No.: US 2012/0245199 A1
(43) Pub. Date: Sep. 27, 2012

(54) [4 [4-(AMINOMETHYL-2-FLUORO-PHENYL)-PIPERIDIN-1-YL]-(1H-PYRROLO-PYRIDIN-YL)-METHANONES AND SYNTHESIS THEREOF

(75) Inventors: Yong Mi CHOI-SLEDESKI, Belle Mead, NJ (US); Thaddeus R. NIEDUZAK, Bridgewater, NJ (US); Gregory B. POLI, Bethlehem, PA (US); Patrick Wai-Kwok SHUM, Flemington, NJ (US); Gregory T. STOKLOSA, Hillsborough, NJ (US); Zhicheng ZHAO, Bridgewater, NJ (US)

(73) Assignee: SANOFI, Paris (FR)

(21) Appl. No.: 13/488,541

(22) Filed: Jun. 5, 2012

Related U.S. Application Data
(63) Continuation of application No. PCT/US2010/060006, filed on Dec. 13, 2010.
(60) Provisional application No. 61/289,565, filed on Dec. 23, 2009.

(30) Foreign Application Priority Data
Sep. 22, 2010 (FR) 1057606

(51) Int. Cl. A61K 31/437; A61P 29/00; C07D 471/04

(52) U.S. Cl. 514/300; 546/113

(57) ABSTRACT

The present invention relates herein to compounds and compositions for the treatment and amelioration of inflammatory disease. Specifically the present invention relates to compounds that having a tryptase inhibition activity and the intermediates thereof, pharmaceutical compositions comprising such compounds, and a method of treating subjects suffering from a condition disease or disorder that can be ameliorated by the administration of an inhibitor of tryptase.

### FIELD OF THE INVENTION

Provided herein are novel and useful compounds having a tryptase inhibition activity and the intermediates thereof, pharmaceutical compositions comprising such compounds, and a method of treating subjects suffering from a condition disease or disorder that can be ameliorated by the administration of an inhibitor of tryptase including but not limited to for example asthma and other inflammatory diseases.

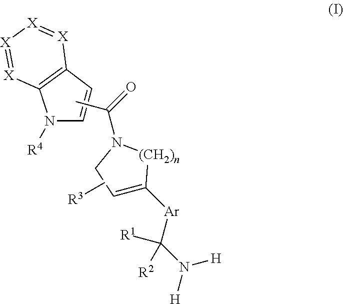

### BACKGROUND OF THE INVENTION

Mast cell mediated inflammatory conditions, in particular asthma, are a growing public health concern. Asthma is frequently characterized by progressive development of hyper-responsiveness of the trachea and bronchi to both immunospecific allergens and generalized chemical or physical stimuli, which lead to the onset of chronic inflammation. Leukocytes containing IgE receptors, notably mast cells and basophils, are present in the epithelium and underlying smooth muscle tissues of bronchi. These leukocytes initially become activated by the binding of specific inhaled antigens to the IgE receptors and then release a number of chemical mediators. For example, degranulation of mast cells leads to the release of proteoglycans, peroxidase, arylsulfatase B, chymase, and tryptase, which results in bronchiole constriction.

Tryptase is stored in the mast cell secretory granules and is the major secretory protease of human mast cells. Tryptase has been implicated in a variety of biological processes, including degradation of vasodilating and bronchorelaxing neuropeptides (Caughey, et al., J. Pharmacol. Exp. Ther., 1988, 244, pages 133-137; Franconi, et al., J. Pharmacol. Exp. Ther., 1988, 248, pages 947-951; and Tam, et al., Am. J. Respir. Cell Mol. Biol., 1990, 3, pages 27-32) and modulation of bronchial responsiveness to histamine (Sekizawa, et al., J. Clin. Invest., 1989, 83, pages 175-179).

As a result, tryptase inhibitors may be useful as anti-inflammatory agents (K Rice, P. A. Sprengler, Current Opinion in Drug Discovery and Development, 1999, 2(5), pages 463-474) particularly in the treatment of chronic asthma (M. Q. Zhang, H. Timmerman, Mediators Inflarnm., 1997, 112, pages 311-317), and may also be useful in treating or preventing allergic rhinitis (S. J. Wilson et al, Clin. Exp. Allergy, 1998, 28, pages 220-227), inflammatory bowel disease (S. C. Bischoff et al, Histopathology, 1996, 28, pages 1-13), psoriasis (A. Naukkarinen et al, Arch. Dermatol. Res., 1993, 285, pages 341-346), conjunctivitis (A. A. Irani et al, J. Allergy Clin. Immunol., 1990, 86, pages 34-40), atopic dermatitis (A. Jarvikallio et al, Br. J. Dermatol., 1997, 136, pages 871-877), rheumatoid arthritis (L. C. Tetlow et al, Ann. Rheum. Dis., 1998, 54, pages 549-555), osteoarthritis (M. G. Buckley et al, J. Pathol., 1998, 186, pages 67-74), gouty arthritis, rheumatoid spondylitis, and diseases of joint cartilage destruction. In addition, tryptase has been shown to be a potent mitogen for fibroblasts, suggesting its involvement in the pulmonary fibrosis in asthma and interstitial lung diseases (Ruoss et al., J. Clin. Invest., 1991, 88, pages 493-499). Therefore, tryptase inhibitors may be useful in treating or preventing fibrotic conditions (J. A. Cairns and A. F. Walls, J. Clin. Invest., 1997, 99, pages 1313-1321) for example, fibrosis, sceleroderma, pulmonary fibrosis, liver cirrhosis, myocardial fibrosis, neurofibromas and hypertrophic scars.

Additionally, tryptase inhibitors may be useful in treating or preventing myocardial infarction, stroke, angina and other consequences of atherosclerotic plaque rupture (M. Jeziorska et al, J. Pathol., 1997, 182, pages 115-122).

Tryptase has also been discovered to activate prostromelysin that in turn activates collagenase, thereby initiating the destruction of cartilage and periodontal connective tissue, respectively.

Therefore, tryptase inhibitors could be useful in the treatment or prevention of arthritis, periodontal disease, diabetic retinopathy, and tumor growth (W. J. Beil et al, Exp. Hematol., 1998 26, pages 158-169). Also, tryptase inhibitors may be useful in the treatment of anaphylaxis (L. B. Schwarz et al, J. Clin. Invest., 1995, 96, pages 2702-2710), multiple sclerosis (M. Steinhoff et al, Nat. Med. (N.Y.), 2000, 6(2), pages 151-158), peptic ulcers and syncytial viral infections.

U.S. Pat. No. 6,977,263 discloses compounds including [(benzylamine)-piperidin-1-yl] (aryl or heteroaryl)methanone as tryptase inhibitors, and describes potential uses for such compounds due to tryptase being implicated in a variety of biological processes, including degradation of vasodilating and bronchorelaxing neuropeptides (Caughey, et al., J. Pharmacol. Exp. Ther., 1988, 244, pages 133, 137; Franconi, et al., J. Pharmacol. Exp. Ther., 1988, 248, pages 947-951; and Tam, et al., Am. J. Respir. Cell Mol. Biol., 1990, 3, pages 27-32) and modulation of bronchial responsiveness to histamine (Sekizawa, et al., J. Clin. Invest., 1989, 83, pages 175-179).

U.S. Pat. No. 6,977,263 more particularly discloses the compounds of formula A, their preparation, and use for treating disease states capable of being modulated by the inhibition

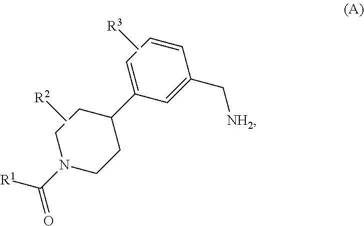

of tryptase. U.S. Pat. No. 6,977,263 also discloses that R¹ of formula A may be an aryl or heteroaryl group. Heteroaryl groups that are exemplified in the U.S. Pat. No. 6,977,263 are alkylpyridyl, alkylthienyl, and indoyl. However there is no teaching or implication that R¹ of formula A may be a pyrrolo-pyridin-yl substituent. Here in we disclose compounds of formula 1 wherein one of the X substituents is a nitrogen (N) thus providing pyrrolo-pyridin-yl compounds with unexpected activity against tryptase.

Accordingly, what is needed is a novel and useful compound having particularly valuable pharmaceutical properties, in its ability to inhibit tryptase. Such a compound should readily have a utility in treating a patient suffering from conditions that can be ameliorated by the administration of an inhibitor of tryptase, e.g., mast cell mediated inflammatory conditions, inflammation, and diseases or disorders related to the degradation of vasodilating and bronchorelaxing neuropeptides.

The present invention further relates to a method for treating or ameliorating macular degeneration in a patient.

Macular degeneration is the general term for a disorder in which a part of the retina called the macula deteriorates. Age-related macular degeneration (AMD) is the most common type of macular degeneration. It has been reported that in the United States, AMD is the leading cause of blindness in people older than 55. More than 10 million people in the US are affected by this dease, which includes 23% of people over 90. (www.webmd.com/eye-health/macular-degeneration/macular-degeneration-overview).

There are various types of macular degeneration that afflict patients. One type of macular degeneration is “dry” macular degeneration. Dry macular degeneration is an early stage of the disorder in which a pigment is deposited on the macula. The deposition of this pigment may result from aging or thinning of the macular tissues. As a result of this deposition of pigment, loss of central vision may gradually occur. Many times, AMD begins with dry macular degeneration.

Another type of AMD is “wet” macular degeneration. Wet macular degeneration is a neovascular type of degeneration in which blood vessels abnormally grow under the retina and begin to leak. As a result of this leakage, permanent damage occurs to light-sensitive cells of the retina which ultimate causes the death of these cells and thus, blind spots. Unlike dry macular degeneration, in which the vision loss may be minor, the vision loss that occurs in wet macular degeneration can be severe. Indeed, it has been reported that although only 10% of those with AMD suffer from wet macular degeneration, 66% of those with AMD suffering from significant visual loss can directly attribute that loss to wet macular degeneration.

Since the causes for macular degeneration are unknown, there has only be limited success determining the causes for the disorder. Moreover, treatments for macular degeneration have met with only limited limited success. To date, there is no FDA-approved treatment for dry macular degeneration and nutritional intervention is used to prevent the progression of wet macular degeneration.

Furthermore, in a method of the present invention, administration of a compound to the patient suffering from macular degeneration modulates the activity of an immunocyte in the patient. The activity of numerous types of immunocytes can be modulated in a method of the present invention. Examples of such immunocytes include a natural killer cell (NK cell), a natural killer T cell (NKT cell), a mast cell, a dendritic cell, and granulocyte selected from the group consisting of an eosinophil, a basophil and neutrophil. Naturally, the activity of a combination of these cells can also be modulated in a method of the present invention.

Moreover, a method of the present invention can also be used to treat or ameliorate choroidal neovascularization, which in turn also treats or ameliorates wet macular degeneration in the patient.

Accordingly, the present invention relates to a methhod of treating a patient in need of amelioration of AMD with a compound of Formula I.

### SUMMARY OF THE INVENTION

The present invention is directed to aminomethyl-2-fluoro-phenyl-piperidin-1-yl]-(1H-pyrrolo-pyridin-yl)-methanones (compounds of formula I)

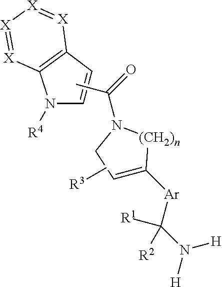

(I); to the syntheses of said compounds and or a prodrug, pharmaceutically acceptable salt, or solvate of said compound to a method of treating patients in need there of. Furthermore, the present invention is directed to a pharmaceutical composition comprising a pharmaceutically effective amount of the compound of formula I, and a pharmaceutically acceptable carrier. Furthermore, the present invention is directed to the use of a compound of formula I as an inhibitor of tryptase, comprising introducing the compound into a composition comprising tryptase. In addition, the present invention is directed to the use of a compound of formula I for treating a patient suffering from, or subject to, a physiological condition in need of amelioration of an inhibitor of tryptase comprising administering to the patient a therapeutically effective amount of the compound of claim 1. The present invention is directed also to the preparation of a compound of formula I.

### DETAILED DESCRIPTION

### Definitions

As used above, and throughout the instant specification and appending claims, the following terms, unless otherwise indicated, shall be understood to have the following meanings:

As used herein, the term “compound of the present invention”, and equivalent expressions, are meant to embrace the compound of formula I, as hereinbefore described, which expression includes the prodrug, the pharmaceutically acceptable salt and the solvate, e.g., hydrate. Similarly, reference to intermediates, whether or not they themselves are claimed, is meant to embrace the salts, and solvates, where the context so permits. For the sake of clarity, particular instances when the context so permits are sometimes indicated in the text, but these instances are purely illustrative and they are not intended to exclude other instances when the context so permits.

As used herein, the term “treatment” or “treating” includes prophylactic therapy as well as treatment of an established condition.

“Patient” means a human or other mammal.

“Effective amount” is meant to describe an amount of a compound effective in producing the desired therapeutic effect.

“Prodrug” means a compound that is suitable for administration to a patient without undue toxicity, irritation, allergic response, and the like, and is convertible in vivo by metabolic means (e.g. by hydrolysis) to the compound of the present invention. A thorough discussion of prodrugs is provided in T. Higuchi and V. Stella, Pro-drugs as Novel Delivery Systems, Vol. 14 of the A. C. S. Symposium Series, and in Edward B. Roche, ed., Bioreversible Carriers in Drug Desiml, American Pharmaceutical Association and Pergamon Press, 1987, both of which are incorporated herein by reference.

### Particular Embodiments

In addition, the present invention is directed to the use of the compound of formula I for treating a patient suffering from a physiological condition that can be ameliorated by administering to the patient a therapeutically effective amount of the compound of formula I. Particular embodiments of physiological conditions that can be treated with the compound of the present invention include, but certainly are not limited to inflammatory diseases, e.g., joint inflammation, arthritis, rheumatoid arthritis, rheumatoid spondylitis, gouty arthritis, traumatic arthritis, rubella arthritis, psoriatic arthritis, and other chronic inflammatory joint diseases. Other embodiments of physiological conditions that can be treated by the present invention include physiological conditions such as chronic obstructive pulmonary disease (COPD), COPD exacerbations, joint cartilage destruction, ocular conjunctivitis, vernal conjunctivitis, inflammatory bowel disease, asthma, allergic rhinitis, interstitial lung diseases, fibrosis, sceleroderma, pulmonary fibrosis, acute macular degneration, macular degeneration, wet macular degeneration, liver cirrhosis, myocardial fibrosis, neurofibromas, hypertrophic scars, various dermatological conditions, for example, atopic dermatitis and psoriasis, myocardial infarction, stroke, angina and other consequences of atherosclerotic plaque rupture, as well as periodontal disease, diabetic retinopathy, tumor growth, anaphylaxis, multiple sclerosis, peptic ulcers, and syncytial viral infections.

In a particular embodiment, the present invention is directed to the use of a compound of formula I for treating a patient suffering from asthma, comprising administering to the patient a physiologically effective amount of the compound.

In another particular embodiment, the present invention is directed to the use of a compound of formula I for treating a patient suffering from COPD, comprising administering to the patient a physiologically effective amount of the compound.

In another particular embodiment, the present invention is directed to the use of a compound of formula I for treating a patient suffering from COPD exacerbations, comprising administering to the patient a physiologically effective amount of the compound.

In another particular embodiment, the present invention is directed to the use of a compound of formula I for treating a patient suffering from allergic rhinitis, comprising administering to the patient a physiologically effective amount of the compound.

In another particular embodiment, the present invention is directed to the use of a compound of formula I for treating a patient suffering from joint inflammation, comprising administering to the patient a physiologically effective amount of the compound.

In another particular embodiment, the present invention is directed to the use of a compound of formula I for treating a patient suffering from macular degeneration, comprising administering to the patient a physiologically effective amount of the compound.

In another particular embodiment, the present invention is directed to the use of a compound of formula I for treating a patient suffering from wet macular degeneration, comprising administering to the patient a physiologically effective amount of the compound.

In another particular embodiment, the present invention is directed to the use of a compound of formula I for treating a patient suffering from acute macular degeneration, comprising administering to the patient a physiologically effective amount of the compound.

In addition, the present invention extends to a pharmaceutical composition comprising the compound of formula I, a second compound selected from the group consisting of a beta andrenergic agonist, an anticholinergic, an anti-inflammatory corticosteroid, and an anti-inflammatory agent, and a pharmaceutically acceptable carrier thereof. In such a composition the compound of formula I and the second compound are present in amounts such that provide a therapeutically efficacious activity, i.e., additive or synergistic effect. Particular inflammatory diseases or disorders that can be treated with such a pharmaceutical composition include, but is not limited to, asthma.

Moreover, the present invention is directed to a method for treating a patient suffering from an inflammatory disorder, comprising administering to the patient the compound of formula I and a second compound selected from the group consisting of a beta andrenergic agonist, an anticholinergic, an anti-inflammatory corticosteroid, and an anti-inflammatory agent. In such a method, the compound of formula I and the second compound are present in amounts such that provide a therapeutically efficacious activity, i.e., additive or synergistic effect. In such a method of the present invention, the compound of the present invention can be administered to the patient before a second compound, a second compound can be administered to the patient before a compound of the present invention, or a compound of the present invention and a second compound can be administered concurrently. Particular examples of andrenergic agonists, anticholinergics, anti-inflammatory corticosteroids, and anti-inflammatory agents having application according to the method are described infra.

### Pharmaceutical Compositions

As explained above, the compound of the present invention exhibits useful pharmacological activity and accordingly may be incorporated into a pharmaceutical composition and used in the treatment of patients suffering from certain medical disorders. The present invention thus provides, according to a further aspect, pharmaceutical compositions comprising the compound of the invention, and a pharmaceutically acceptable carrier thereof. As used herein, the term “pharmaceutically acceptable” preferably means approved by a regulatory agency of a government, in particular the Federal government or a state government, or listed in the U.S. Pharmacopoeia or another generally recognized pharmacopoeia for use in animals, and more particularly in humans. Suitable pharmaceutical carriers are described in “Remington's Pharmaceutical Sciences” by E. W. Martin. Pharmaceutical compositions according to the present invention can be prepared according to the customary methods, using one or more pharmaceutically acceptable adjuvants or excipients. The adjuvants comprise, inter alia, diluents, fillers, binders, disintegrants, glidants, lubricants, surfactants, sterile aqueous media and the various non-toxic organic solvents. The composition may be presented in the form of tablets, capsules, pills, sustained release formulations, granules, powders, aqueous solutions or suspensions, injectable solutions, elixirs or syrups, and can contain one or more agents chosen from the group comprising sweeteners, flavorings, colorings, or stabilizers in order to obtain pharmaceutically acceptable preparations. The choice of vehicle and the content of active substance in the vehicle are generally determined in accordance with the solubility and chemical properties of the active compound, the particular mode of administration and the provisions to be observed in pharmaceutical practice. For example, excipients such as lactose, microcrystalline cellulose, pregelatinized starch, unmodified starch, silicified microcrystalline cellulose, mannitol, sorbitol, xylitol, dextrates, fructose, sodium citrate, calcium carbonate, dicalcium phosphate dihydrate, anhydrous dicalcium phosphate, calcium sulfate, along with binders such as polyvinylpyrollidone, hydroxypropylmethyl cellulose, ethyl cellulose, hydroxyethyl cellulose, methyl cellulose, sodium carboxymethyl cellulose, pregelatinized starch, starch, polyethylene glycols, polyethylene oxide, polycarbophils, gelatin and acacia and disintegrating agents such as sodium croscannellose, sodium starch glycolate, crospovidone, starch, microcrystalline cellulose, alginic acids and certain complex silicates combined with lubricants such as magnesium stearate, calcium stearate, stearic acid, hydrogenated vegetable oil, mineral oil, polyethylene glycols, glyceryl esters of fatty acids, sodium lauryl sulfate and glidants such as silicon dioxide, talc, starch, along with some suitable wetting agent such as sodium lauryl sulfate, sorbitan esters, polyoxyethylene fatty acid esters, poloxamer, polyoxyethylene ether, sodium docusate, polyethoxylated castor oil, and benzalkonium chloride may be used for preparing tablets. To prepare a capsule, it is advantageous to use fillers such as lactose, microcrystalline cellulose, pregelatinized starch, unmodified starch, silicified microcrystalline cellulose alone or a mixture of two or more fillers, with and without binders as described above along with suitable wetting agent (s), disintegrants, glidants, lubricants, etc. as listed above. When aqueous suspensions are used they can contain emulsifying agents or agents which facilitate suspension.

Diluents such as sucrose, ethanol, polyethylene glycol, propylene glycol, glycerol and chloroform or mixtures thereof may also be used. Such pharmaceutically acceptable carriers can also be sterile water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include mannitol, human serum albumin (HSA), starch, glucose, lactose, sucrose, gelatin, malt, rice, flour, chalk, silica gel, magnesium carbonate, magnesium stearate, sodium stearate, glycerol monostearate, talc, sodium chloride, dried skim milk, glycerol, propylene, glycol, water, ethanol and the like. These compositions can take the form of solutions, suspensions, tablets, pills, capsules, powders, sustained-release formulations and the like.

Naturally, a pharmaceutical composition of the present invention will contain a therapeutically effective amount of the active compound together with a suitable amount of carrier so as to provide the form for proper administration to the patient. While intravenous injection is a very effective form of administration, other modes can be employed, such as by injection, or by oral, nasal or parenteral administration, which are discussed infra.

### Methods of Treatment

The compound of formula I possesses tryptase inhibition activity according to tests described in the literature and described hereinafter, and which test results are believed to correlate to pharmacological activity in humans and other mammals. Thus, in a further embodiment, the present invention is directed to the use of formula I or a composition comprising it for treating a patient suffering from, or subject to, a condition that can be ameliorated by the administration of an inhibitor of tryptase. For example, the compound of formula I is useful for treating an inflammatory disease, for example, joint inflammation, including arthritis, rheumatoid arthritis and other arthritic condition such as rheumatoid spondylitis, gouty arthritis, traumatic arthritis, rubella arthritis, psoriatic arthritis, osteoarthritis or other chronic inflammatory joint disease, or diseases of joint cartilage destruction, ocular conjunctivitis, vernal conjunctivitis, inflammatory bowel disease, asthma, allergic rhinitis, interstitial lung diseases, fibrosis, sceleroderma, pulmonary fibrosis, liver cirrhosis, myocardial fibrosis, neurofibromas, hypertrophic scars, various dermatological conditions, for example, atopic dermatitis and psoriasis, myocardial infarction, stroke, angina or other consequences of atherosclerotic plaque rupture, as well as periodontal disease, diabetic retinopathy, macular degeneration, acute macular degeneration, wet, macular degeneration, tumor growth, anaphylaxis, multiple sclerosis, peptic ulcers, or a syncytial viral infection.

According to a further feature of the invention there is provided a method for the treatment of a human or animal patient suffering from, or subject to, conditions which can be ameliorated by the administration of an inhibitor of tryptase, for example conditions as hereinbefore described, which comprises the administration to the patient of an effective amount of compound of the invention or a composition containing a compound of the invention.

### Combination Therapy

As explained above, other pharmaceutically active agents can be employed in combination with the compound of formula I depending upon the disease being treated. For example, in the treatment of asthma, beta-adrenergic agonists such as albuterol, terbutaline, formoterol, fenoterol or prenaline can be included, as can anticholinergics such as ipratropium bromide, anti-inflammatory corticosteroids such as beclomethasone dipropionate, triamcinolone acetonide, flunisolide or dexamethasone, and anti-inflammatory agents such as sodium cromoglycate and nedocromil sodium. Thus, the present invention extends to a pharmaceutical composition comprising the compound of formula I and a second compound selected from the group consisting of a beta andrenergic agonist, an anticholinergic, an anti-inflammatory corticosteroid, and an anti-inflammatory agent; and a pharmaceutically acceptable carrier thereof. Particular pharmaceutical carriers having applications in this pharmaceutical composition are described herein.

Furthermore, the present invention extends to a method for treating a patient suffering from asthma, comprising administering the patient the compound of the present invention, and a second compound selected from the group consisting of a beta andrenergic agonist, an anticholinergic, an anti-inflammatory corticosteroid, and an anti-inflammatory agent. In such a combination method, the compound of the present invention can be administered prior to the administration of the second compound, the compound of the present invention can be administered after administration of the second compound, or the compound of the present invention and the second compound can be administered concurrently.

### Modes of Delivery

According to the invention, the compound of formula I, or a pharmaceutical composition comprising the compound, may be introduced parenterally, transmucosally, e.g., orally, nasally, intraocularly, pulmonarily, or rectally, or transdermally to a patient.

### Oral Delivery

Contemplated for use herein are oral solid dosage forms, which are described generally in Remington's Pharmaceutical Sciences, 18th Ed. 1990 (Mack Publishing Co. Easton Pa. 18042) at Chapter 89, which is herein incorporated by reference. Solid dosage forms include tablets, capsules, pills, troches or lozenges, cachets or pellets. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). A description of possible solid dosage forms for a therapeutic is given by Marshall, K. In: Modern Pharmaceutics Edited by G. S. Banker and C. T. Rhodes Chapter 10, 1979, herein incorporated by reference. In general, the formulation will include a compound of the present invention, and inert ingredients that allow for protection against the stomach environment, and release of the biologically active material, i.e., a compound of the present invention, in the intestine.

Also specifically contemplated are oral dosage forms of the compound of the present invention. Such a compound may be chemically modified so that oral delivery is more efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the compound of the present invention, and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189. Other polymers that could be used are poly-1,3-dioxolane and poly-1,3,6-tioxocane. Preferred for pharmaceutical usage, as indicated above, are polyethylene glycol moieties.

For the compound of the present invention, the location of release may be the stomach, the small intestine (the duodenum, the jejunum, or the ileum), or the large intestine. One skilled in the art has available formulations that will not dissolve in the stomach, yet will release the material in the duodenum or elsewhere in the intestine. Preferably, the release will avoid the deleterious effects of the stomach environment, either by protection of the compound of the present invention, or by release of the compound beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings may be used as mixed films.

A coating or mixture of coatings can also be used on tablets, which are not intended for protection against the stomach. This can include sugar coatings, or coatings that make the tablet easier to swallow. Capsules may consist of a hard shell (such as gelatin) for delivery of dry therapeutic i.e. powder; for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the compound of the present invention may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, alpha-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include, but are not limited to starch, including the commercial disintegrant based on starch, Explotab sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carhoxymethyl cellulose. These surfactants could be present in the formulation of a compound of the present invention either alone or as a mixture in different ratios.

Additives that potentially enhance uptake of the compound of the present invention are, for instance, the fatty acids oleic acid, linoleic acid and linolenic acid. Controlled release oral formulation may be desirable. The drug could be incorporated into an inert matrix that permits release by either diffusion or leaching mechanisms, e.g., gums. Slowly degenerating matrices may also be incorporated into the formulation. Some enteric coatings also have a delayed release effect.

Another form of a controlled release of this therapeutic is by a method based on the Oros therapeutic system (Alza Corp.), i.e. the drug is enclosed in a semipermeable membrane which allows water to enter and push drug out through a single small opening due to osmotic effects.

Other coatings may be used for the formulation. These include a variety of sugars that could be applied in a coating pan. The therapeutic agent could also be given in a film-coated tablet and the materials used in this instance are divided into 2 groups. The first are the non-enteric materials and include methyl cellulose, ethyl cellulose, hydroxyethyl cellulose, methylhydroxyethyl cellulose, hydroxypropyl cellulose, hydroxypropyl-methyl cellulose, sodium carboxy-methyl cellulose, providone and the polyethylene glycols. The second group consists of the enteric materials that are commonly esters of phthalic acid.

A mix of materials might be used to provide the optimum film coating. Film coating may be carried out in a pan-coater or in a fluidized bed or by compression coating.

### Pulmonary Delivery

Also contemplated herein is pulmonary delivery of the compound of the present invention, either alone, or in a pharmaceutical composition. The compound is delivered to the lungs of a mammal while inhaling and traverses across the lung epithelial lining to the blood stream. Other reports of this include Adjei et al., 1990, Pharmaceutical Research, 7:565-569; Adjei et al., 1990, International Journal of Pharmaceutics, 63: 135-144 (leuprolide acetate); Braquet et al., 1989, Journal of Cardiovascular Pharmacology, 13(suppl. 5): 143-146 (endothelin-1); Hubbard et al., 1989, Annals of Internal Medicine, Vol. III, pp. 206-212 (al-antitrypsin); Smith et al., 1989, J. Clin. Invest. 84: 1145-1146 (a-1-proteinase); Oswein et al., 1990, “Aerosolization of Proteins”, Proceedings of Symposium on Respiratory Drug Delivery 11, Keystone, Colo., March, (recombinant human growth hormone); Debs et al., 1988, J. Immunol. 140:3482-3488 (interferon-y and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Wallinckrodt, Inc., St. Louis, Mo.; the Acorn I1 nebulizer, manufactured by Marquest Medical Products, Englewood, Colo.; the Ventolin metered dose inhaler, manufactured by Glaxo Inc., Reseaxch Triangle Park, North Carolina; and the Spinhaler powder inhaler, manufactured by Fisons Corp., Bedford, Mass., to name only a few. All such devices require the use of formulations suitable for the dispensing of the compound of the present invention. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. A chemically modified compound of the present invention may also be prepared in different formulations depending on the type of chemical modification or the type of device employed.

Formulations suitable for use with a nebulizer, either jet or ultrasonic, will typically comprise the compound of the present invention dissolved in water at a concentration of about 0.1 to 25 mg of compound per mL of solution. The formulation may also include a buffer and a simple sugar (e.g., for stabilization and regulation of osmotic pressure). The nebulizer formulation may also contain a surfactant, to reduce or prevent surface induced aggregation of the compound caused by atomization of the solution in forming the aerosol.

Formulations for use with a metered-dose inhaler device will generally comprise a finely divided powder containing the compound of the invention suspended in a propellant with the aid of a surfactant. The propellant may be any conventional material employed for this purpose, such as a chlorofluorocarbon, hydrochlorofluorocarbon, hydrofluorocarbon, a r hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing the compound of the invention, and may also include a bulking agent, such as lactose, sorbitol, sucrose, or mannitol in amounts which facilitate dispersal of the powder from the device, e.g., 50 to 90% by weight of the formulation. The compound of the present invention should most advantageously be prepared in particulate form with an average particle size of less than 10 mm (or microns), most preferably 0.5 to 5 mm, for most effective delivery to the distal lung.

### Nasal Delivery

Nasal delivery of the compound of the present invention is also contemplated. Nasal delivery allows the passage of the compound to the blood stream directly after administering the therapeutic product to the nose, without the necessity for deposition of the product in the lung. Formulations for nasal delivery include those with dextran or cyclodextran.

### Intraocular Delivery

Intraocular delivery of the compound of the present invention is also contemplated. Various and numerous methods are known in the art for intraoculal administration of a drug. Intraocular delivery allows the passage of the compound to the intaocular fluid directly after administering the therapeutic product to the eye, without the necessity for oral administration of the product. Formulations for intaocular delivery may include, but are not limited to, solutions or suspensions in aqueous or non-aqueous media.

### Transdermal Delivery

Various and numerous methods are known in the art for transdermal administration of a drug, e.g., via a transdermal patch. Transderma administration methods have applications in the present invention. Transdermal patches are described in for example, U.S. Pat. No. 5,407,713, issued Apr. L 8, 1995 to Rolando et al.; U.S. Pat. No. 5,352,456, issued Oct. 4, 1994 to Fallon et al.; U.S. Pat. No. 5,332,213 issued Aug. 9, 1994 to D'Angelo et al.; U.S. Pat. No. 5,336,168, issued Aug. 9, 1994 to Sibalis; U.S. Pat. No. 5,290,561, issued Mar. 1, 1994 to Farhadieh et al.; U.S. Pat. No. 5,254,346, issued Oct. 19, 1993 to Tucker et al.; U.S. Pat. No. 5,164,189, issued Nov. 17, 1992 to Berger et al.; U.S. Pat. No. 5,163,899, issued Nov. 17, 1992 to Sibalis; U.S. Pat. Nos. 5,088,977 and 5,087,240, both issued Feb. 18, 1992 to Sibalis; U.S. Pat. No. 5,008,110, issued Apr. 16, 1991 to Benecke et al.; and U.S. Pat. No. 4,921,475, issued May 1, 1990 to Sibalis, the disclosure of each of which is incorporated herein by reference in its entirety.

It can be readily appreciated that a transdermal route of administration may be enhanced by use of a dermal penetration enhancer, e.g., such as enhancers described in U.S. Pat. No. 5,164,189 (supra), U.S. Pat. No. 5,008,110 (supra), and U.S. Pat. No. 4,879,119, issued Nov. 7, 1989 to Aruga et al., the disclosure of each of which is incorporated herein by reference in its entirety.

### Topical Administration

For topical administration, gels (water or alcohol based), creams or ointments containing compounds of the invention may be used. Compounds of the invention may also be incorporated in a gel or matrix base for application in a patch, which would allow a controlled release of compound through the transdermal barrier.

### Rectal Administration

Solid compositions for rectal administration include suppositories formulated in accordance with known methods and containing the compound of the invention.

### Dosages

The percentage of active ingredient in the composition of the invention may be varied, it being necessary that it should constitute a proportion such that a suitable dosage shall be obtained. Obviously, several unit dosage forms may be administered at about the same time. The dose employed will be determined by the physician, and depends upon the desired therapeutic effect, the route of administration and the duration of the treatment, and the condition of the patient. In the adult, the doses are generally from about 0.001 to about 50, preferably about 0.001 to about 5, mg per kg body weight per day by inhalation, from about 0.01 to about 100, preferably 0.1 to 70, more especially 0.5 to 10, mg per kg body weight per day by oral administration, and from about 0.001 to about 10, preferably 0.01 to 1, mg per kg body weight per day by intravenous administration. In each particular case, the doses will be determined in accordance with the factors distinctive to the subject to be treated, such as age, weight, general state of health and other characteristics which can influence the efficacy of the medicinal product.

Furthermore, the compound according to the invention may be administered as frequently as necessary in order to obtain the desired therapeutic effect. Some patients may respond rapidly to a higher or lower dose and may find much weaker maintenance doses adequate. For other patients, it may be necessary to have long-term treatments at the rate of 1 to 4 doses per day, in accordance with the physiological requirements of each particular patient. Generally, the active product may be administered orally 1 to 4 times per day. Of course, for some patients, it will be necessary to prescribe not more than one or two doses per day.

Naturally, a patient in whom administration of the compound of the present invention is an effective therapeutic regimen is preferably a human, but can be any animal. Thus, as can be readily appreciated by one of ordinary skill in the art, the methods and pharmaceutical compositions of the present invention are particularly suited to administration to any animal, particularly a mammal, and including, but by no means limited to, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

### Preparatory Details

The compound of formula I may be prepared by the application or adaptation of known methods, by which is meant methods used heretofore or described in the literature, for example those described by R. C. Larock in Comprehensive Organic Transformations, VCH publishers, 1989, or as described herein.

In the reactions described hereinafter it may be necessary to protect reactive functional groups, for example, amino groups, to avoid their unwanted participation in the reactions. Conventional protecting groups may be used in accordance with standard practice, for examples see T. W. Greene and P.G.M.Wuts in “Protective Groups in Organic Chemistry” John Wiley and Sons, 1991. In particular, the compound of formula I may be prepared as shown through Examples 1-20 below. For example, the compound of the present invention is an achiral compound whose preparation is comprised of a convergent synthesis.

As used throughout the specification, the following abbreviations and definitions, unless otherwise indicated, shall be understood to have the following meanings:

### List of Abbreviations

APCI atmospheric pressure chemical ionization

BOC tert-butyl dicarbonate

BOC anhydride di-tert-butyl dicarbonyl anhydride

t-Bu tert-butyl

t-BuOH tert-butanol

CDCl₃ deuterated chloroform

CD₃OD deuterated methanol

DCM dichloromethane, CH₂Cl₂ or methylenechloride

DMAP 4-dimethylaminopyridine

DMF dimethylformamide

DMSO dimethylsulfoxide

DMSO-d₆ dimethyl-d₆ sulfoxide

dppf 1,1′-bis(diphenylphosphino)ferrocene

eq equivalent(s)

Et ethyl

Et₂O diethyl ether

Et₃N triethylamine

EtOH ethanol

EtOAc ethyl acetate

EDCI 1-ethyl-3-(3′-dimethylaminopropyl)carbodiimide

HPLC high performance liquid chromatography

H₂ hydrogen

L Liter

LC/MS liquid chromatography-mass spectrometry

M molar

Me methyl

MeCN acetonitile

MeOH methanol

MgSO₄ magnesium sulfate

MHz megahertz

min minute

OMe methoxide

NaHCO₃ sodium bicarbonate

Na₂CO₃ sodium carbonate

NaCl sodium chloride

NaOH sodium hydroxide

NaI sodium iodide

NaOMe sodium methoxide

Na₂SO₄ sodium sulfate

n-BuOAc n-butyl acetate

NMR nuclear magnetic resonance

Pd/C Palladium on carbon

Pd(PPh₃)₄ tetrakistriphenylphosphine palladium

Pd(PPh₃)₂Cl₂ bistriphenylphosphine palladium (II) dichloride

PdCl₂dppf 1,1′-bis(diphenylphosphino) ferrocene palladium (II) dichloride

Pd(dtbpf)Cl₂ (1,1′-bis(di-t-butylphosphino)ferrocene palladium dichloride

Pd₂(dba)₃ tris(dibenzylideneacetone)dipalladium(0)

Pd(OAc)₂ palladium(II) acetate

P(Cy)₃ tricyclohexylphosphine

t-Bu₃P tri-t-butylphosphine

PPh₃ triphenylphosphine

PrOH propanol

i-PrOH iso-propanol

Pt/C platinum on carbon

t-BuOK potassium tert-butoxide

rt room temperature

Rt Retention time

sat saturated

SiO₂ silica

TFA trifluoroacetic acid

THF tetrahydrofuran

TLC thin layer chromatography

TMS trimethylsilyl

### Terms

### Preparatory Details

The starting materials for preparing compound I according to Scheme 1 below are commercially available.

### Example 1

### [4-(5-Aminomethyl-2-fluoro-phenyl)-piperidin-1-yl]-(1H-pyrrolo[2,3-b]pyridin-2-yl)-methanone dihydrochloride

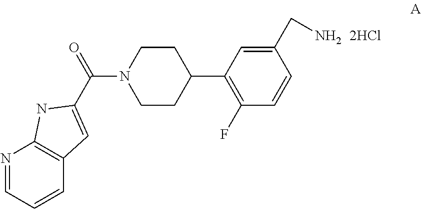

### A. 2,2,2-Trifluoro-N-(4-fluoro-3-pyridin-4-yl-benzyl)-acetamide hydrochloride

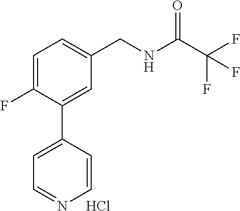

A flask is charged with NaHCO₃ (126 g, 1.5 mol), 3-bromo-4-fluorobenzylamine hydrochloride (12, 120 g, 0.5 mole) and pyridine-4-boronic acid (13, 67.6 g, 0.55 mmol) and isopropyl alcohol (750 mL) and water (375 mL) at room temperature. The suspension is degassed with N₂ for 1.0 h at 10° C. Into the mixture is added 1,1′-bis(diphenylphosphino)ferrocene-palladium(II)dichloride dichloromethane complex (PdCl₂dppf-CH₂Cl₂, 16.4 g, 20 mmol). The reaction mixture is ramped to 80° C. while some part is distilled off until the internal temperature reached to 80° C., and stirred for 10 h. After the reaction is completed (HPLC analysis), the mixture is cooled to room temperature, and aqueous 2 N HCl (750 mL) is added, and stirred for 0.5 h. The solution is washed with CH₂Cl₂ (750 mL and 500 mL). To the aqueous phase is charged 50% aqueous NaOH (100 mL) to adjust pH>13. After adding n-BuOAc (2.0 L), activated carbon (50 g) is added into the organic layer. This mixture is filtered through a pad of celite (50 g). Azeotropic distillation is performed. After adding an additional n-BuOAc (1.0 L), the reaction mixture is cooled to 5° C. Trifluoroacetic anhydride (157 g, 0.6 mol) is slowly added into the solution at 5° C. After the reaction is completed (HPLC analysis), the reaction mixture is washed with aqueous 10% Na₂CO₃ (1.0 L). A solution of 5-6 N HCl in isopropanol (120 mL) is introduced into the crude organic layer at 10° C. Additional n-BuOAc (1.0 L) is then added, the suspension is left overnight at room temperature. The resultant solid is filtered at 10° C., and dried in oven at 50° C. to give the desired product (124 g, 75%) as a white solid: mp=220° C. Anal. Calcd for C₁₄H₁₀F₄N₂O—HCl: C, 50.24; H, 3.31; N, 8.37. Found: C, 50.16; H, 3.08; N, 8.38. MS (ESI) m/z 299 (M+H). ¹H NMR (300 MHz, D₂O) δ 8.70 (d, J=6.9 Hz, 2H), 8.14 (d, J=6.9 Hz, 2H), 7.56-7.20 (m, 3H), 4.51 (s, 2H).

### B. 2,2,2-trifluoro-N-(4-fluoro-3-piperidin-4-yl-benzyl)-acetamide hydrochloride

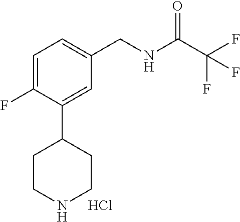

A Parr flask is charged with 2,2,2-trifluoro-N-(4-fluoro-3-pyridin-4-yl-benzyl)-acetamide hydrochloride (123 g, 0.37 mol) and MeOH (740 mL) at room temperature. Then, 5% Pt/C (36.9 g, 30 w/w %) is added. The reaction flask is placed in a Parr hydrogenation system and charged with H₂ at 50-60 psi. The mixture is shaken for >48 h while charging H₂ until the pressure reached a steady state (H₂ was refilled to 50-60 psi every 2-3 hours during day time while 10-20 psi is observed without any further refill after overnight). When HPLC analysis shows completion of the reaction, the reaction mixture is filtered through a pad of Celite. The filtrate is distilled at 40-50° C. while adding n-BuOAc (1.25 L). After completion of distillation of MeOH, additional n-BuOAc (1 L) is added. The resultant suspension is allowed to cool to rt overnight. The suspension is cooled to 10° C., filtered, and dried in oven at 50° C. to give 112 g (89%) of desired product as white solid: mp=134° C. Anal. Calcd for C₁₄H₁₀F₄N₂O—HCl: C, 50.24; H, 3.31; N, 8.37. Found: C, 50.16; H, 3.08; N, 8.38. MS (ESI) m/z 305.4 (M+H). ¹H NMR (300 MHz, D₂O) δ 7.16-6.98 (m, 3H), 4.34 (s, 2H), 3.42 (d, J=12.9 Hz, 2H), 3.14-2.99 (m, 3H), 1.98-1.81 (m, 4H).

### C. 2,2,2-Trifluoro-N-{4-fluoro-3-[1-(1H-pyrrolo[2,3-b]pyridine-2-carbonyl)-piperidin-4-yl]-benzyl}-acetamide

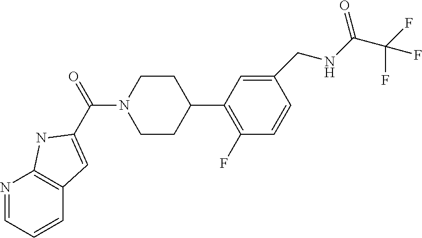

To a suspension of 1H-pyrrolo[2,3-b]pyridine-2-carboxylic acid (0.43 g, 2.59 mmol), 2,2,2-trifluoro-N-(4-fluoro-3-piperidin-4-yl-benzyl)-acetamide hydrochloride (Example 1B, 0.88 g, 2.59 mmol), and EDCI (0.88 g, 3.11 mmol) in delete CH₂Cl₂(50 mL) is added Et₃N (0.865 mL, 6.22 mmol). The reaction is stirred at room temperature overnight. The reaction mixture is poured into EtOAc and the organic layer is washed with sat NH₄Cl, water and brine. The organic is dried over MgSO₄, filtered and concentrated in vacuo to give the crude product. Purification by flash chromatography on SiO₂ eluting with 100% EtOAc gives 0.95 g, (82%) of the desired product.

¹H-NMR (CDCl₃, 300 MHz) δ 10.4 (s, H), 8.5 (d, H), 8.0 (d, H), 7.2-7.0 (m, 4H), 6.8 (s, H), 6.7 (bs, H), 4.8 (m, 2H), 4.5 (d, 2H), 3.4-3.0 (m, 3H), 2.7-2.0 (m, 4H). LCMS m/z 449 (M+H).

### D. [4-(5-Aminomethyl-2-fluoro-phenyl)-piperidin-1-yl]-(1H-pyrrolo[2,3-b]pyridin-2-yl)-methanone dihydrochloride

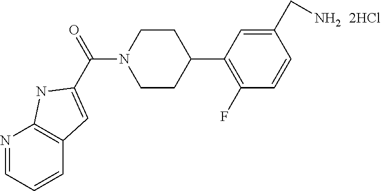

To a solution 2,2,2-trifluoro-N-{4-fluoro-3-[1-(1H-pyrrolo[2,3-b]pyridine-2-carbonyl)-piperidin-4-yl]-benzyl}-acetamide (0.78 g, 1.74 mmol) in 30 ml MeOH and H₂O (12 mL) was added K₂CO₃ (2.4 g, 17.4 mmol). The reaction mixture is stirred overnight. The reaction mixture is concentrated in vacuo to remove most of the methanol. The residue is partitioned between H₂O and EtOAc washed with H₂O and brine, dried over MgSO₄, filtered, and concentrated in vacuo. The residue is taken up in 4.0 N HCl/Dioxane (10 mL, 40.0 mmol) and stirred 10 min The reaction mixture is concentrated in vacuo and Et₂O (20 mL) is added. A solid precipitate forms and the ethereal solution is decanted off. The solid is washed with additional Et₂O and then isolated by filtration to give 0.31 g (46%) of the desired product. ¹H-NMR (DMSO-d6, 300 MHz) δ 12.2 (s, H), 8.4 (s, H), 8.2 (bs, 2H), 8.1 (d, H), 7.5 (d, H), 7.4 (m, H), 7.25 (m, H), 7.19 (m, H), 6.8 (s, H), 4.6 (m, 2H), 4.2-3.8 (m, 3H), 3.2 (m, 2H), 1.85 (m, 2H), 1.7 (m, 2H). LCMS m/z 353 (M+H).

### Example 2

### [4-(5-Aminomethyl-2-fluoro-phenyl)-piperidin-1-yl]-[1-(2-methoxy-ethyl)-1H-pyrrolo[2,3-b]pyridin-2-yl]-methanone dihydrochloride

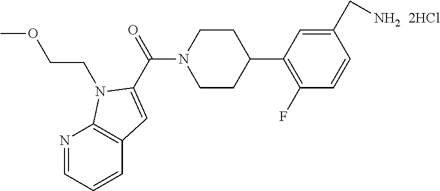

### A. 1H-Pyrrolo[2,3-b]pyridine-2-carboxylic acid ethyl ester

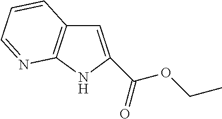

A solution of 1H-pyrrolo[2,3-b]pyridine-2-carboxylic acid (20 g, 123 mmol), H₂SO₄ (20 mL) and EtOH is heated to reflux for 4 h. The reaction mixture is concentrated in vacuo, taken up in EtOAc and washed with sat. NaHCO₃ (2×), H₂O and brine, dried over MgSO₄, filtered, and concentrated in vacuo to yield 14 g (60%) of desired product. ¹H-NMR (CDCl₃, 300 MHz) δ 11.6 (bs, H), 8.6 (d, H), 8.1 (d, H), 7.2 (m, 2H), 4.45 (m, 2H), 1.5 (m, 3H). LCMS m/z 191 (M+H).

### B. 1-(2-Methoxy-ethyl)-1H-pyrrolo[2,3-b]pyridine-2-carboxylic acid ethyl ester

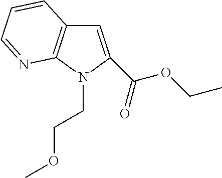

To a suspension of NaH (0.51 g, 12.63 mmol) in DMF (10 mL) under Ar is added a suspension of 1H-pyrrolo[2,3-b]pyridine-2-carboxylic acid ethyl ester (2 g, 10.53 mmol) in DMF (9 mL). The reaction mixture is stirred for 20 min, and 2-methoxyethyl bromide (1.1 mL, 11.8 mmol) is added. The reaction mixture is stirred at room temperature overnight. The reaction mixture is poured into H₂O and the resulting precipitate isolated by filtration. Purification by flash chromatography on SiO₂ eluting with 10% EtOAc gives 1.27 g, (49%) of the desired product. ¹H-NMR (CDCl₃, 300 MHz) δ 8.5 (d, H), 8.0 (d, H), 7.2 (s, H), 7.15 (m, H), 5.0 (t, 2H), 4.4 (t, 2H), 3.75 (t, 2H), 3.3 (s, 3H), 1.4 (t, 3H). LCMS m/z 249 (M+H).

### C. 1-(2-Methoxy-ethyl)-1H-pyrrolo[2,3-b]pyridine-2-carboxylic acid

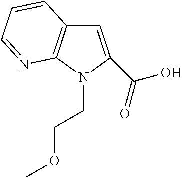

1-(2-Methoxy-ethyl)-1H-pyrrolo[2,3-b]pyridine-2-carboxylic acid ethyl ester (1 g, 4.03 mmol), EtOH (30 mL), THF (10 mL) and 1N NaOH solution (16 mL) are heated to 50° C. for 1 h and then stirred at room temperature overnight. The reaction is cooled to room temperature, acidified to pH=3 with concentrated HCl and extracted with EtOAc (3×). The organic fractions are combined, dried over MgSO₄, filtered, and concentrated in vacuo to yield 0.73 g (83%) of desired product. ¹H-NMR (DMSO-d6, 300 MHz) δ 13.2 (bs, H), 8.45 (d, H), 8.1 (d, H), 7.2 (m, 2H), 4.85 (t, 2H), 3.6 (t, 2H), 3.2 (s, 3H). LCMS m/z 221 (M+H).

### D. 2,2,2-Trifluoro-N-(4-fluoro-3-{1-[1-(2-methoxy-ethyl)-1H-pyrrolo[2,3-b]pyridine-2-carbonyl]-piperidin-4-yl}-benzyl)-acetamide

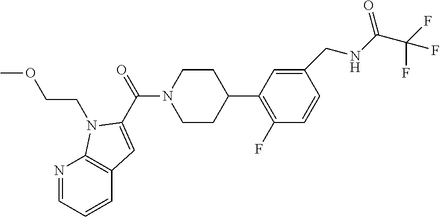

The title compound is prepared in a similar manner as Example 1C using 1-(2-methoxy-ethyl)-1H-pyrrolo[2,3-b]pyridine-2-carboxylic acid as the starting material. ¹H NMR (CDCl₃, 300 MHz) δ 8.4 (d, H), 7.9 (d, H), 7.2-7.0 (m, 5H), 6.55 (s, 2H), 4.75 (m, 2H), 4.5 (m, 2H), 3.7 (t, 2H), 3.25 (s, 3H), 2.0-1.8 (m, 4H), 1.55 (m, 4H). LCMS m/z 507 (M+H).

### E. [4-(5-Aminomethyl-2-fluoro-phenyl)-piperidin-1-yl]-[1-(2-methoxy-ethyl)-1H-pyrrolo[2,3-b]pyridin-2-yl]-methanone dihydrochloride

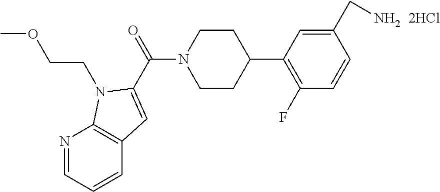

The title compound is prepared in a similar manner as Example 1D using 1,2,2-trifluoro-N-(4-fluoro-3-{1-[1-(2-methoxy-ethyl)-1H-pyrrolo[2,3-b]pyridine-2-carbonyl]-piperidin-4-yl}-benzyl)-acetamide as the starting material. ¹H NMR (DMSO-d6, 300 MHz) δ 8.4 (d, H), 8.2 (bs, 2H), 8.0 (d, H), 7.6 (d, H), 7.35 (m, H), 7.3-7.1 (m, 2H), 6.7 (s, 1H), 4.6 (bm, 2H) 4.0 (bm, 2H), 3.6 (m, 5H), 3.25-3.15 (m, 5H), 2.0-1.6 (m, 4H). MS m/z 411 (M+H).

### Example 3

### [4-(3-Aminomethyl-phenyl)-piperidin-1-yl]-[1-(2-methoxy-ethyl)-1H-pyrrolo[2,3-b]pyridin-2-yl]-methanone dihydrohydrochloride

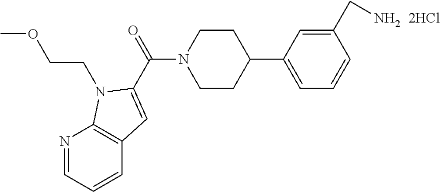

### A. 4-Oxo-piperidine-1-carboxylic acid 2-trimethylsilanyl-ethyl ester

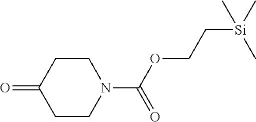

A solution of 4-piperidone monohydrate hydrochloride (25 g, 88.22 mmol), 2-trimethylsilylethyl p-nitrophenylcarbonate (50 mL, 359.70 mmol), triethylamine (50 mL, 345.00 mmol) and DMAP (10.78 g, 88.24 mmol) in of acetonitrile (300 mL) is warmed under reflux for 2 hours and then allowed to cool to room temperature. The mixture is diluted with dichloromethane (300 mL) and washed 1 M HCl (3×100 mL) and 1 M NaOH (4×100 mL) until all of the yellow color is removed from the organic phase. The organic phase is then washed with brine and dried over MgSO₄. The organic phase is concentrated in vacuo to afford 19.35 g (90%) of the title compound as a colorless oil. ¹H NMR (CDCl₃, 300 MHz) δ 4.22 (m, 2H), 3.75 (t, J=6.2 Hz, 4H), 2.44 (t, J=6.2 Hz, 4H), 1.02 (m, 2H), 0.04 (s, 9H).

### B. 4-(3-Cyanophenyl)-3,6-dihydro-2H-pyridine-1-carboxylic acid 2-trimethylsilanyl-ethyl

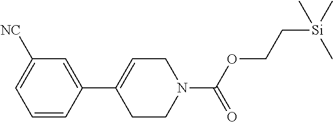

To a flask containing tetrahydrofuran (50 mL) at −70° C. is added 1M lithium hexamethyldisilazide (60 mL, 60 mmol) dropwise. A solution of 4-oxo-piperidine-1-carboxylic acid 2-trimethylsilanyl-ethyl ester (13.3 g, 55 mol) is then added via dropping funnel over 20 minutes keeping the internal temperature between −65° C. and −70° C. The solution is stirred at −70° C. for 45 minutes then a solution of phenyltrifluoromethane sulfonamide (19.65 g, 55 mmol) in THF (75 mL) is added dropwise over 20 minutes. The solution is allowed to warm to 0° C. and stirred for 3 hours. The reaction is then concentrated in vacuo and the residue, 4-trifluoromethanesulfonyloxy-3,6-dihydro-2H-pyridine-1-carboxylic acid 2-trimethyl-silanyl-ethyl ester, is used without further purification. To a solution of 4-trifluoromethanesulfonyloxy-3,6-dihydro-2H-pyridine-1-carboxylic acid 2-trimethyl-silanyl-ethyl ester (20.65 g, 55 mmol) acetonitrile (300 mL) is added 3-cyanophenylboronic acid (8.9 g (60.6 mmol) followed by 2 M sodium carbonate (82.5 mL 165 mmol), lithium chloride (6.98 g, 165 mmol) and tetrakistriphenylphosphine palladium (0) (3.18 g, 2.8 mmol). The mixture is warmed under reflux for 90 minutes then allowed to cool to room temperature and filtered. The filtrate is concentrated and diluted 2 M Na₂CO₃ (300 mL) then extracted 3× dichloromethane. The organic phase is washed with brine then separated and dried (MgSO₄). The organic phase is concentrated in vacuo and the crude residue is flash chromatographed over SiO₂ (eluted with heptane:EtOAc:DCM=5:1:1) to give 10.46 g (58%) of the title compound as a yellow oil. ¹H NMR (CDCl₃, 300 MHz) δ 7.65-7.52 (m, 3H), 7.44 (t, J=7.7 Hz, 1H), 6.11 (bs, 1H), 4.23 (m, 2H), 4.15 (m, 2H), 3.70 (t, J=5.6 Hz, 2H), 2.52 (m, 2H), 1.04 (m, 2H), 0.06 (s, 9H).

### C. 4-(3-Aminomethyl-phenyl)-piperidine-1-carboxylic acid 2-trimethylsilanyl-ethyl ester

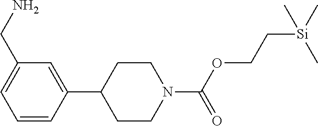

To a slurry of 10% Pd/C (5 g, wet) in ethanol (250 mL) is added concentrated HCl (2.9 mL, 34.8 mmol) and 4-(3-cyanophenyl)-3,6-dihydro-2H-pyridine-1-carboxylic acid 2-trimethylsilanyl-ethyl ester (10.4 g). The mixture is hydrogentated at 50 psi for 4 hours. The mixture is then filtered over a cake of Celite and the cake is washed with excess ethanol. The filtrate is then concentrated in vacuo and the residue is triturated with Et₂O/pentane, then filtered to give 7.1 g of the title compound as a white solid. ¹H NMR (CD₃OD, 300 MHz) δ 7.41-7.27 (m, 4H), 4.26 (dm, J=13.5 Hz, 2H), 4.20 (m, 2H), 4.09 (s, 2H), 2.92 (bm, 2H), 2.79 (tt, J=12.1, 3.6 Hz, 1H), 1.84 (dm, J=12.9 Hz, 2H), 1.62 (qd, J=12.6, 4.1 Hz, 2H), 1.02 (m, 2H), 0.06 (s, 9H); MS (APCI) m/z 336, 335 (M+H, 100), 191.

### D. 4-[3-(tert-Butoxycarbonylamino-methyl)-phenyl]-piperidine-1-carboxylic acid 2-trimethylsilanyl-ethyl ester

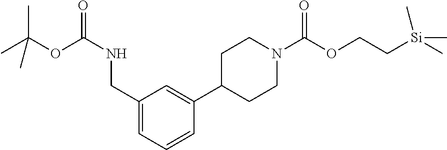

To a solution of 4-(3-aminomethyl-phenyl)-piperidine-1-carboxylic acid 2-trimethylsilanyl-ethyl ester (11.1 g, 29.93 mmol) in dichloromethane (150 mL) and saturated NaHCO₃ (50 mL) is added Boc-anhydride (6.54 g, 29.96 mmol.). The mixture is stirred overnight at room temperature. The organic phase is then separated and washed with water and brine. The organic phase is then separated, dried (MgSO₄) and concentrated in vacuo to give 13.41 g (100%) of the title compound as an oil. ¹H NMR (CDCl₃, 300 MHz) δ 7.26 (m, 1H), 7.10 (m, 3H), 4.85 (bs, 1H), 4.29 (d, J=5.8 Hz, 4H), 4.19 (m, 2H), 2.83 (t, J=12.5 Hz, 2H), 2.64 (tt, J=12.0, 3.6 Hz, 1H), 1.81 (m, 2H), 1.60 (m, 2H), 1.45 (s, 9H), 1.01 (t, J=8.4 Hz, 2H), 0.04 (s, 9H).

### E. (3-Piperidin-4-yl-benzyl)-carbamic acid tert-butyl ester

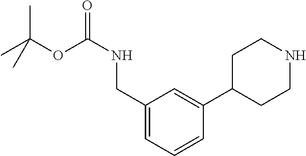

To a solution of 4-(3-tert-butoxycarbonylaminomethylphenyl)-piperidine-1-carboxylic acid 2-trimethylsilanyl-ethyl ester (13.41 g (30.9 mmol) tetrahydrofuran (200 mL) is added tetrabutyl ammonium fluoride (1M in THF, 34 mL, 34 mmol). The mixture is warmed to 50° C. for 2 hours then allowed to cool to room temperature and stand overnight. To complete the reaction the mixture is heated for an additional 3 h at 50° C. The mixture is then concentrated in vacuo, diluted with 1M HCl and extracted with Et₂O. The aqueous phase is made basic with 1N NaOH and extracted 3× with EtOAc. The organic phases are combined, washed with brine, separated and dried (MgSO₄). The organic phase is filtered and concentrated in vacuo to afford 8.3 g (93%) of the title compound as a yellow oil which is used without further purification. ¹H NMR (CDCl₃, 300 MHz) δ 7.25 (m, 1H), 7.07-7.13 (m, 3H), 4.85 (bs, 1H), 4.29 (d, J=5.1 Hz, 2H), 3.17 (dm, J=12.0 Hz, 2H), 2.72 (td, J=12.0, 2.4 Hz, 2H), 2.60 (tt, J=12.0, 3.6 Hz, 1H), 1.81 (m, 2H), 1.55-1.70 (m, 3H), 1.46 (s, 9H). LCMS m/z 291 (M+H).

### F. (3-{1-[1-(2-Methoxy-ethyl)-1H-pyrrolo[2,3-b]pyridine-2-carbonyl]-piperidin-4-yl}-benzyl)-carbamic acid tert-butyl ester

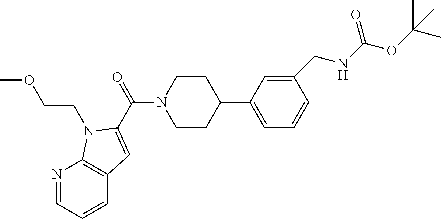

The title compound is prepared in a similar manner as Example 1C using 1-(2-methoxy-ethyl)-1H-pyrrolo[2,3-b]pyridine-2-carboxylic acid and (3-piperidin-4-yl-benzyl)-carbamic acid tert-butyl ester as the starting material. ¹H NMR (CDCl₃, 300 MHz) δ 8.4 (d, 1H), 7.95 (d, 1H), 7.3 (m, 2H), 7.2-7.0 (m, 4H), 6.55 (s, 1H), 4.9 (bs, 1H), 4.7 (m, 3H), 4.3 (m, 2H), 3.7 (t, 2H), 3.3 (s, 3H), 2.8 (m, 1H), 1.95 (m, 2H), 1.8 (m, 3H), 1.5 (m, 1H), 1.45 (s, 9H). LCMS m/z 493 (M+H).

### G. [4-(3-Aminomethyl-phenyl)-piperidin-1-yl]-[1-(2-methoxy-ethyl)-1H-pyrrolo[2,3-b]pyridin-2-yl]-methanone dihydrochloride

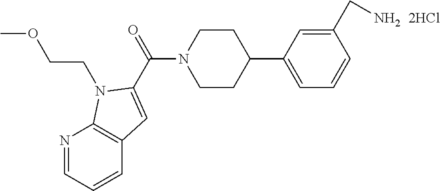

3-{1-[1-(2-Methoxy-ethyl)-1H-pyrrolo[2,3-b]pyridine-2-carbonyl]-piperidin-4-yl}-benzyl)-carbamic acid tert-butyl ester (0.33 g, 0.66 mmol) and 4.0 N HCl/dioxane (8 mL, 32 mmol) are stirred for 3 h. The reaction mixture is concentrated in vacuo and Et₂O (20 mL) is added. A solid precipitate forms and the ethereal solution is decanted off. The solid is washed with additional Et₂O and then isolated by filtration to give 0.28 g (98%) of the desired product. ¹H-NMR (DMSO-d6, 300 MHz) δ 8.4 (d, 1H), 8.3 (bs, 2H), 8.1 (d, 1H), 7.45-7.3 (m, 4H), 7.2 (m, H), 6.7 (s, 1H), 4.8-4.5 (m, 3H), 4.2 (m, 4H), 4.0 (m, 2H), 3.2 (s, 3H), 2.9 (m, 2H), 2.0-1.6 (m, 4H). LCMS m/z 393 (M+H).

### Example 4

### [4-(5-Aminomethyl-2-fluoro-phenyl)-piperidin-1-yl]-[1-(2-methoxy-ethyl)-1H-pyrrolo[3,2-c]pyridin-2-yl]-methanone hydrochloride

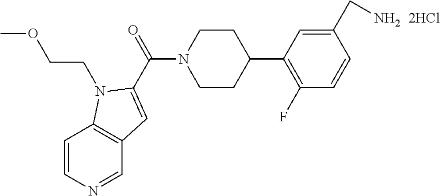

### A. 1H-Pyrrolo[3,2-c]pyridine-2-carboxylic acid methyl ester

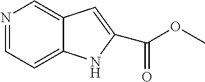

To a solution of 1H-pyrrolo[3,2-c]pyridine-2-carbaldehyde (3.24 g, 22.19 mmol) in MeOH at 0° C. under argon is added sodium cyanide (5.44 g, 111 mmol) and manganese dioxide (9.65 g, 111 mmol). The reaction mixture is stirred for 5 h after which time it is filtered through Celite and diluted with EtOAc (500 mL). The organic layer is washed with water (2×), brine, dried over sodium carbonate, filtered and concentrated to yield 3.27 g (84%) of desired product. ¹H NMR (DMSO-d6, 300 MHz) δ 12.3 (bs, 1H), 9.0 (s, 1H), 8.3 (d, 1H), 7.4 (d, 1H), 7.3 (s, 1H), 4.0 (s, 3H). LCMS m/z 177 (M+H).

### B. 1-(2-Methoxy-ethyl)-1H-pyrrolo[3,2-c]pyridine-2-carboxylic acid methyl ester

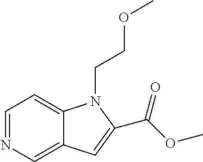

The title compound is prepared in a similar manner as Example 2B using 1H-pyrrolo[3,2-c]pyridine-2-carboxylic acid methyl ester as the starting material. ¹H NMR (CDCl₃, 300 MHz) δ 9.0 (s, 1H), 8.2 (bs, 2H), 8.4 (d, 1H), 7.4 (m, 2H), 4.7 (t, 2H), 4.0 (s, 3H), 3.8 (t, 2H), 3.3 (s, 3H). LCMS m/z 235 (M+H).

### C. 1-(2-Methoxy-ethyl)-1H-pyrrolo[3,2-c]pyridine-2-carboxylic acid

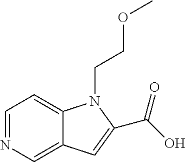

To 1-(2-methoxy-ethyl)-1H-pyrrolo[3,2-c]pyridine-2-carboxylic acid methyl ester (0.18 g, 0.77 mmol) MeOH (15 mL) is added 1 N NaOH (5 mL). The resulting solution is stirred at room temperature overnight. The reaction mixture is acidified to pH=2 with 1 N HCl and is washed with EtOAc. The aqueous layer is lypholized to dryness and the resulting solid triturated with MeOH. The MeOH layer is concentrated in vacuo to yield 0.165 g (97%) of desired product. ¹H-NMR (CD₃OD, 300 MHz) δ 13.2 (bs, 1H), 9.3 (s, 1H), 8.4 (d, 1H), 8.2 (d, 1H), 7.8 (s, 1H), 4.95 (t, 2H), 3.8 (t, 2H), 3.2 (s, 3H). LCMS m/z 221 (M+H).

### D. 2,2,2-Trifluoro-N-(4-fluoro-3-{1-[1-(2-methoxy-ethyl)-1H-pyrrolo[3,2-c]pyridine-2-carbonyl]-piperidin-4-yl}-benzyl)-acetamide

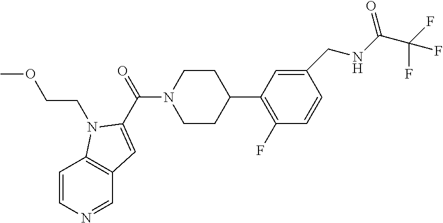

The title compound is prepared in a similar manner as Example 1C using 1-(2-methoxy-ethyl)-1H-pyrrolo[3,2-c]pyridine-2-carboxylic acid as the starting material.

¹H NMR (CDCl₃, 300 MHz) δ 8.9 (s, 1H), 8.4 (d, 1H), 7.35 (d, 1H), 7.2 (m, 2H), 6.7 (s, 1H), 4.5 (m, 4H), 3.7 (t, 2H), 3.3 (s, 3H), 3.2 (m, 2H), 1.9 (m, 2H), 1.8 (m, 2H), 1.6 (bs, 4H). LCMS m/z 507 (M+H).

### E. [4-(5-Aminomethyl-2-fluoro-phenyl)-piperidin-1-yl]-[1-(2-methoxy-ethyl)-1H-pyrrolo[3,2-c]pyridin-2-yl]-methanone dihydrochloride

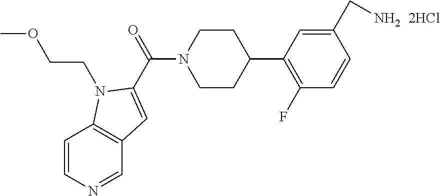

The title compound is prepared in a similar manner as Example 1D using 2,2,2-trifluoro-N-(4-fluoro-3-{1-[1-(2-methoxy-ethyl)-1H-pyrrolo[3,2-c]pyridine-2-carbonyl]-piperidin-4-yl}-benzyl)-acetamide as the starting material.

¹H NMR (DMSO-d6, 300 MHz) δ 9.35 (s, 1H), 8.6 (d, 1H), 8.4 (bs, 2H), 8.2 (d, H), 7.6 (d, 1H), 7.4 (m, 1H), 7.3-7.1 (m, 2H), 4.65 (m, 3H), 4.0 (m, 5H), 3.6 (t, 2H), 3.2 (s, 3H), 3.0 (m, H), 1.9 (m, 2H), 1.8 (m, 2H). LCMS m/z 411 (M+H).

### Example 5

### [4-(5-Aminomethyl-2-fluoro-phenyl)-piperidin-1-yl]-(1H-pyrrolo[2,3-c]pyridin-3-yl)-methanone dihydrochloride

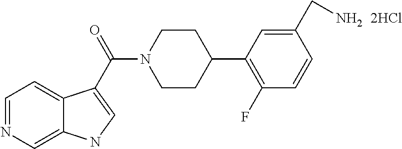

### A. 2,2,2-Trichloro-1-(1H-pyrrolo[2,3-c]pyridin-3-yl)-ethanone

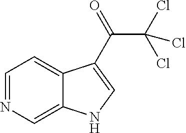

To 1H-pyrrolo[2,3-c]pyridine (5 g, 42.4 mmol) in CH₂Cl₂ (150 mL) is added aluminum chloride (42.4 g, 318 mmol). The reaction mixture is heated to 48° C. and trichloroacetyl chloride (8.1 g, 44.5 mmol) is added dropwise. After heating for 2 h, the reaction mixture is cooled to 0° C. and is quenched with 200 mL H₂O and the resulting precipitate is isolated by filtration to give 10 g of the desired product (89%). ¹H NMR (DMSO-d6, 300 MHz) δ 9.3 (s, 1H), 9.25 (s, 1H), 8.6 (m, 2H). LCMS m/z 263 (M+H), 265.

### B. 1H-Pyrrolo[2,3-c]pyridine-3-carboxylic acid

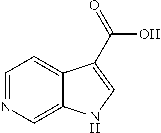

A mixture of 2,2,2-trichloro-1-(1H-pyrrolo[2,3-c]pyridin-3-yl)-ethanone (6.7 g, 25.4 mmol) and 6N NaOH (150 mL) is heated at reflux for 3 h and then 110° C. overnight. The reaction mixture is diluted with H₂O (200 mL), washed with CH₂Cl₂ (2×) and acidified to pH=2 with conc. HCl. The aqueous layer is lyophilized to dryness and the resulting solid triturated with MeOH. The MeOH layer is concentrated in vacuo to yield 3.5 g (85%) of desired product. ¹H-NMR (DMSO-d6, 300 MHz) δ 10.0 (s, 1H), 8.9 (s, 1H), 8.35-8.25 (m, 2H), 8.2 (s, 1H), 8.0 (m, 1H). LCMS m/z 163 (M+H).

### C. 2,2,2-Trifluoro-N-{4-fluoro-3-[1-(1H-pyrrolo[2,3-c]pyridine-3-carbonyl)-piperidin-4-yl]-benzyl}-acetamide

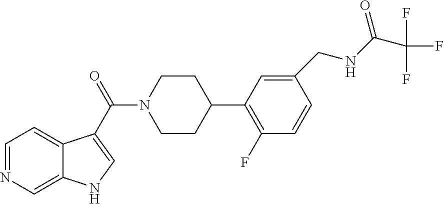

The title compound is prepared in a similar manner as Example 1C using 1H-pyrrolo[2,3-c]pyridine-3-carboxylic acid as the starting material. ¹H NMR (DMSO-d6, 300 MHz) δ 13.4 (s, 1H), 10.0 (m, 1H), 9.3 (bs, 1H), 8.6 (s, 1H), 8.4 (bs, 1H), 8.2 (bs, 1H), 7.3 (m, 1H), 7.2 (d, 2H), 4.4 (m, 3H), 3.2 (m, 3H), 1.8-1.6 (m, 5H). LCMS m/z 449 (M+H).

### D. [4-(5-Aminomethyl-2-fluoro-phenyl)-piperidin-1-yl]-(1H-pyrrolo[2,3-c]pyridin-3-yl)-methanone dihydrochloride

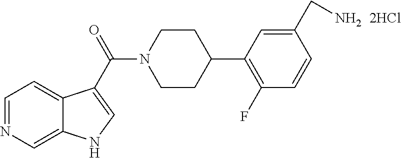

To a solution 2,2,2-trifluoro-N-{4-fluoro-3-[1-(1H-pyrrolo[2,3-c]pyridine-3-carbonyl)-piperidin-4-yl]-benzyl}-acetamide (0.58 g, 1.29 mmol) in MeOH (40 mL) and H₂O (17 mL) is added K₂CO₃ (1.79 g, 12.9 mmol). The reaction mixture is stirred overnight. The reaction is partitioned between H₂O and EtOAc washed with H₂O and brine, dried over MgSO₄, filtered, and concentrated in vacuo. The residue is taken up in Et₂O (10 mL) and 2.0 N HCl/Et₂O (15 mL, 30.0 mmol) is added. A solid precipitate forms and the ethereal solution is decanted off. The solid is washed with additional Et₂O and then isolated by filtration to give 0.2 g (40%) of the desired product. ¹H-NMR (DMSO-d6, 300 MHz) δ 13.5 (s, 1H), 9.2 (s, 1H), 8.6 (s, 1H), 8.5 (d, 1H), 8.4 (bs, 2H), 8.2 (d, 1H), 7.6 (m, 1H), 7.4 (m, 1H), 7.2 (m, 1H), 4.4 (bs, 1H), 4.0 (m, 2H), 3.2 (m, 3H), 1.85 (m, 2H), 1.7 (m, 2H), 1.2 (m, 1H). LCMS m/z 353 (M+H).

### Example 6

### [4-(5-Aminomethyl-2-fluoro-phenyl)-piperidin-1-yl]-[1-(2-methoxy-ethyl)-1H-pyrrolo[3,2-b]pyridin-2-yl]-methanone dihydrochloride

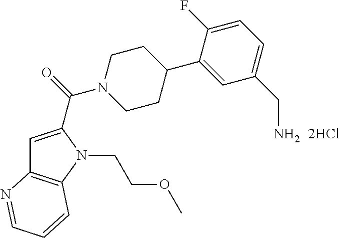

### A. 1-(2-Methoxy-ethyl)-1H-pyrrolo[3,2-b]pyridine-2-carboxylic acid ethyl ester

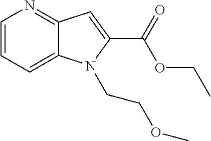

To a solution of 1H-pyrrolo[3,2-b]pyridine-2-carboxylic acid ethyl ester (1.34 g, 7.04 mmol) [prepared according to the procedure by Lachance, N. et al. Synthesis 2005, 15, 2571-2577] in N,N-dimethylacetamide at rt is added sodium hydride (210 mg, 8.31 mmol). The resulting mixture is stirred for 30 minutes at rt. 2-Bromoethylmethyl ether (1.4 mL, 14.15 mmol) is added and the resulting mixture is stirred at rt overnight. The mixture is diluted with water and EtOAc. The organic is separated and the aqueous phase is extracted with EtOAc. The organic phase is washed with brine then separated and dried (MgSO₄). The organic phase is concentrated in vacuo and the crude residue is flash chromatographed over SiO₂ (eluted with heptane:EtOAc=85:15) to afford 1.24 g (71%) of the title compound as a yellow oil. ¹H NMR (CDCl₃, 300 MHz) δ 8.56 (m, 1H), 7.82 (d, 1H), 7.46 (s, 1H), 7.24-7.20 (m, 1H), 4.73 (m, 2H), 4.40 (m, 2H), 3.73 (m, 2H), 3.24 (m, 3H), 1.43 (m, 3H). LCMS m/z 249 (M+H).

### B. 1-(2-Methoxy-ethyl)-1H-pyrrolo[3,2-b]pyridine-2-carboxylic acid trifluoroacetate

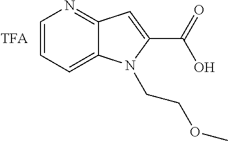

To a solution of 1-(2-methoxy-ethyl)-1H-pyrrolo[3,2-b]pyridine-2-carboxylic acid ethyl ester (1.24 g, 5.00 mmol) in a mixture of THF:MeOH:H₂O (1:1:1) (30 mL) is added lithium hydroxide hydrate (1.1 g, 26.12 mmol). The resulting mixture is stirred for one hour. The mixture is acidified with 2 N HCl to pH 2-3. The solvents are removed in vacuo and the aqueous phase is flash freeze and lyophilized. The solid is flash chromatographed over reverse phase (C₁₈) (eluted with 10% MeCN/0.1% TFA in H₂O to 100% MeCN on a 25 minutes ramp) to afford 1.66 g (99% as a TFA salt) of the title compound as a white solid. ¹H NMR (CD₃OD, 300 MHz) δ 8.83 (d, 1H), 8.73 (d, 1H), 7.82 (dd, 1H), 7.48 (s, 1H), 5.00 (t, 2H), 3.77 (t, 2H), 3.22 (s, 3H). LCMS m/z 221 (M+H).

### C. 2,2,2-Trifluoro-N-(4-fluoro-3-{1-[1-(2-methoxy-ethyl)-1H-pyrrolo[3,2-b]pyridine-2-carbonyl]-piperidin-4-yl}-benzyl)-acetamide

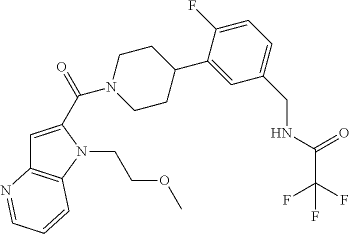

To a solution of 1-(2-methoxy-ethyl)-1H-pyrrolo[3,2-b]pyridine-2-carboxylic acid (434 mg, 1.30 mmol) in dichloromethane (25 mL) and N,N-dimethylformamide (1 mL) is added 1-(3-dimethylaminopropyl)-3-ethylcarbodiimide hydrochloride (295 mg, 1.54 mmol), 1-Hydroxybenzotriazole (194 mg, 1.44 mmol) and triethylamine (550 μL, 3.93 mmol). The resulting mixture is stirred for 20 minutes at rt. 2,2,2-trifluoro-N-(4-fluoro-3-piperidin-4-yl-phenyl)-acetamide hydrochloride (466 mg, 1.37 mmol) is added and heated at 40° C. overnight. The mixture is poured into water and the organic layer separated. The aqueous phase is extracted with EtOAc (×3). The organic phases are washed with brine then separated and dried (MgSO₄). The organic phase is concentrated in vacuo and the crude residue is flash chromatographed over SiO₂ (eluted with MeOH:CH₂Cl₂=3:97) to afford 369 mg (56%) of the title compound as a white solid. ¹H NMR (CD₃OD, 300 MHz) δ 8.40 (m, 1H), 8.03 (d, 1H), 7.32-7.28 (m, 2H), 7.22-7.17 (m, 1H), 7.09-7.02 (m, 1H), 6.80 (s, 1H), 4.56 (m, 2H), 4.43 (s, 2H), 4.32 (bs, 2H), 3.65 (m, 2H), 3.24 (s, 5H), 3.07-2.99 (m, 2H), 1.89-1.77 (m, 4H). LCMS m/z 507 (M+H).

### D. [4-(5-Aminomethyl-2-fluoro-phenyl)-piperidin-1-yl]-[1-(2-methoxy-ethyl)-1H-pyrrolo[3,2-b]pyridin-2-yl]-methanone dihydrochloride

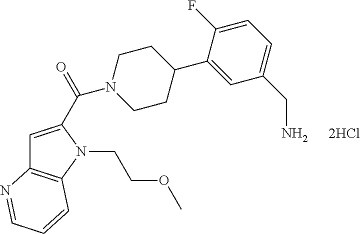

The title compound is prepared in a similar manner as Example 1D using 2,2,2-trifluoro-N-(4-fluoro-3-{1-[1-(2-methoxy-ethyl)-1H-pyrrolo[3,2-b]pyridine-2-carbonyl]-piperidin-4-yl}-benzyl)-acetamide as the starting material. ¹H NMR (DMSO-d6, 300 MHz) δ 8.76-8.71 (m, 2H), 8.42 (bs, 3H), 7.70 (dd, 1H), 7.66-7.63 (m, 1H), 7.43-7.38 (m, 1H), 7.26-7.20 (m, 1H), 7.06 (s, 1H), 4.71-4.63 (m, 3H), 4.02-3.93 (m, 3H), 3.62 (t, 2H) 3.56 (s, 3H), 3.55-3.45 (m, 2H), 3.11-2.94 (m, H), 1.94-1.72 (m, 4H). LCMS m/z 411 (M+H).

### Example 7

### [4-(3-Aminomethyl-phenyl)-piperidin-1-yl]-[1-(2-methoxy-ethyl)-1H-pyrrolo[3,2-b]pyridin-2-yl]-methanone dihydrochloride

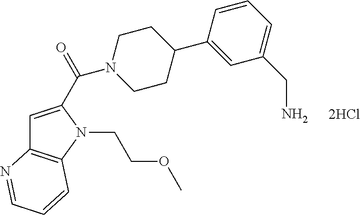

### A. (3-{1-[1-(2-Methoxy-ethyl)-1H-pyrrolo[3,2-b]pyridine-2-carbonyl]-piperidin-4-yl}-benzyl)-carbamic acid tert-butyl ester

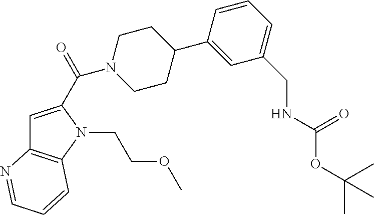

The title compound is prepared in a similar manner as Example 6C using 1-(2-methoxy-ethyl)-1H-pyrrolo[3,2-b]pyridine-2-carboxylic acid and (3-piperidin-4-yl-benzyl)-carbamic acid tert-butyl ester as the starting materials. ¹H NMR (CD₃OD, 300 MHz) δ 8.40 (d, 1H), 8.02 (d, 1H), 7.32-7.11 (m, 5H), 6.80 (s, 1H), 4.55 (t, 2H), 4.22 (br s, 3H), 3.65 (t, 2H), 3.24 (s, 3H), 3.00-2.87 (m, 3H), 1.86-1.72 (m, 3H), 1.45 (br s, 9H), 1.39-1.29 (m, 2H), 0.87 (br s, 1H). LCMS m/z 493 (M+H).

### B. [4-(3-Aminomethyl-phenyl)-piperidin-1-yl]-[1-(2-methoxy-ethyl)-1H-pyrrolo[3,2-b]pyridin-2-yl]-methanone dihydrochloride

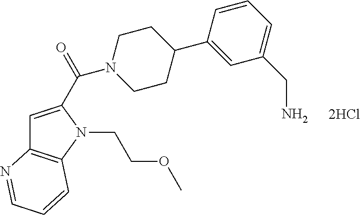

The title compound is prepared in a similar manner as Example 3G using (3-{1-[1-(2-methoxy-ethyl)-1H-pyrrolo[3,2-b]pyridine-2-carbonyl]-piperidin-4-yl}-benzyl)-carbamic acid tert-butyl ester as the starting material. ¹H NMR (DMSO-d6, 300 MHz) δ 8.40 (d, 1H), 8.77-8.71 (m, 2H), 8.40 (br s, 3H), 7.72 (dd, H), 7.48 (s, H), 7.41-7.31 (m, 3H), 7.08 (s, H), 4.64 (m, 2H), 4.00 (m, 3H), 3.62 (t, 2H), 3.57 (s, 3H), 3.49 (m, H), 3.03-2.87 (m, H), 1.96-1.67 (m, 4H). LCMS m/z 393 (M+H).

### Example 8

### [4-(3-Aminomethyl-phenyl)-piperidin-1-yl]-[1-(2-methoxy-ethyl)-1H-pyrrolo[2,3-c]pyridin-3-yl]-methanone dihydrochloride

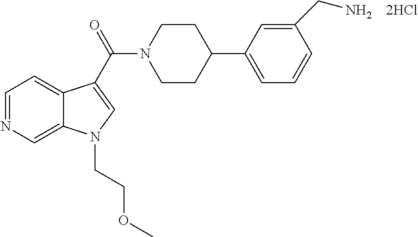

### A. 1H-Pyrrolo[2,3-c]pyridine-3-carboxylic acid methyl ester

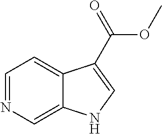

To a solution of 2,2,2-trichloro-1-(1H-pyrrolo[2,3-c]pyridin-3-yl)-ethanone (7.8 g, 29.6 mmol) in 90 mL MeOH is added a 30% wt solution NaOMe/MeOH (10 mL, 177 mmol). The reaction mixture is stirred for 2 h and is then concentrated in vacuo. The residue is taken up in EtOAc, washed with H₂O and brine, dried over MgSO₄, filtered, and concentrated in vacuo to yield 4 g (77%) of desired product. ¹H NMR (CDCl₃, 300 MHz) δ 8.85 (s, 1H), 8.4 (d, 1H), 8.1 (m, 2H), 3.95 (s, 3H). LCMS m/z 177 (M+H).

### B. 1-(2-Methoxy-ethyl)-1H-pyrrolo[2,3-c]pyridine-3-carboxylic acid methyl ester

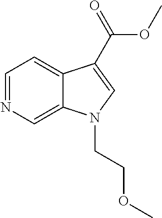

The title compound is prepared in a similar manner as Example 2B using 1H-pyrrolo[2,3-c]pyridine-3-carboxylic acid methyl ester as the starting material. ¹H NMR (CDCl₃, 300 MHz) δ 8.8 (s, 1H), 8.4 (m, 1H), 8.0 (m, 2H), 4.4 (t, 2H), 3.9 (s, 3H), 3.75 (t, 2H), 3.3 (s, 3H). LCMS m/z 235 (M+H).

### C. 1-(2-Methoxy-ethyl)-1H-pyrrolo[2,3-c]pyridine-3-carboxylic acid

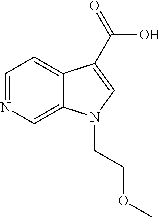

To 1-(2-methoxy-ethyl)-1H-pyrrolo[2,3-c]pyridine-3-carboxylic acid methyl ester (2 g, 8.5 mmol) in MeOH (25 mL) is added 1N NaOH (31 mL). The resulting solution is stirred at room temperature overnight. The reaction mixture is acidified to pH=3 with 1 N HCl and is concentrated in vacuo to remove the MeOH. A solid precipitate forms and is isolated by filtration to give 1.64 g (88% yield) of desired product. ¹H-NMR (DMSO-d6, 300 MHz) δ 13.0 (bs, 1H), 9.5 (s, 1H), 8.8 (s, 1H), 8.5 (d, 1H), 8.4 (d, 1H), 4.7 (t, 2H), 3.8 (t, 2H), 3.2 (s, 3H). MS m/z 221 (M+H).

### D. (3-{1-[1-(2-Methoxy-ethyl)-1H-pyrrolo[2,3-c]pyridine-3-carbonyl]-piperidin-4-yl}-benzyl)-carbamic acid tert-butyl ester

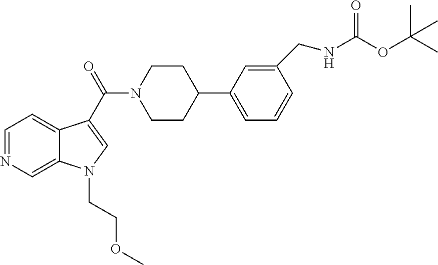

The title compound is prepared in a similar manner as Example 1C using 1-(2-methoxy-ethyl)-1H-pyrrolo[2,3-c]pyridine-3-carboxylic acid and (3-piperidin-4-yl-benzyl)-carbamic acid tert-butyl ester as the starting materials. The material is used in the next step without further purification.

### E. [4-(3-Aminomethyl-phenyl)-piperidin-1-yl]-[1-(2-methoxy-ethyl)-1H-pyrrolo[2,3-c]pyridin-3-yl]-methanone dihydrochloride

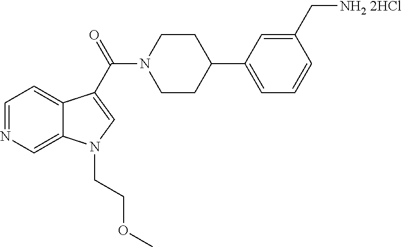

A solution of (3-{1-[1-(2-methoxy-ethyl)-1h-pyrrolo[2,3-c]pyridine-3-carbonyl]-piperidin-4-yl}-benzyl)-carbamic acid tert-butyl ester (0.896 g, 1.82 mmol) in a saturated HCl/EtOAc solution (30 mL) is stirred at room temperature for 4 h. The resulting precipitate is isolated by filtration to give 0.77 g (98%) of desired product. ¹H NMR (DMSO-d6, 300 MHz) δ 9.5 (s, 1H), 8.8 (s, 1H), 8.45 (d, H), 8.4 (bs, 2H), 8.2 (d, 1H), 7.45 (s, 1H), 7.35 (m, 3H), 4.7 (t, 2H), 4.0 (m, 2H), 3.75 (t, 2H), 3.4 (m, 4H), 3.2 (s, 3H), 2.9 (m, 1H), 1.9 (m, 2H), 1.75 (m, 2H). LCMS m/z 393 (M+H).

### Example 9

### [4-(5-Aminomethyl-2-fluoro-phenyl)-piperidin-1-yl]-[1-(2-methoxy-ethyl)-1H-pyrrolo[2,3-c]pyridin-3-yl]-methanone dihydrochloride

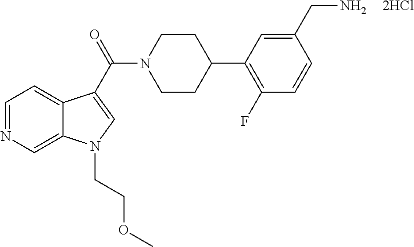

### A. 2,2,2-Trifluoro-N-(4-fluoro-3-{1-[1-(2-methoxy-ethyl)-1H-pyrrolo[2,3-c]pyridine-3-carbonyl]-piperidin-4-yl}-benzyl)-acetamide

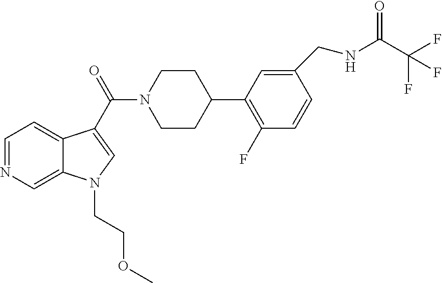

The title compound is prepared in a similar manner as Example 1C using 1-(2-methoxy-ethyl)-1H-pyrrolo[2,3-c]pyridine-3-carboxylic acid as the starting material. ¹H-NMR (CDCl₃, 300 MHz) δ 9.4 (s, 1H), 8.35 (d, 1H), 8.15 (d, 1H), 8.1 (s, 1H), 7.2 (m, 2H), 7.1 (m, 1H), 6.7 (bs, 1H), 6.7 (bs, 1H), 4.5 (m, 6H), 3.8 (t, 2H), 3.3 (s, 3H), 3.2 (m, 3H), 2.0 (m, 2H), 1.8 (m, 2H). LCMS m/z 507 (M+H)

### B. [4-(5-Aminomethyl-2-fluoro-phenyl)-piperidin-1-yl]-[1-(2-methoxy-ethyl)-1H-pyrrolo[2,3-c]pyridin-3-yl]-methanone dihydrochloride

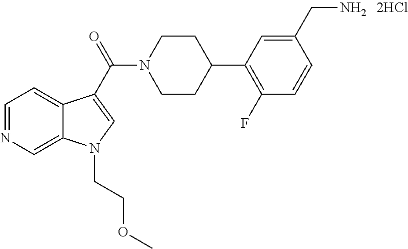

The title compound is prepared in a similar manner as Example 5D using 2,2,2-trifluoro-N-(4-fluoro-3-{1-[1-(2-methoxy-ethyl)-1H-pyrrolo[2,3-c]pyridine-3-carbonyl]-piperidin-4-yl}-benzyl)-acetamide as the starting material. ¹H-NMR (DMSO-d6, 300 MHz) δ 9.4 (s, 1H), 8.5 (s, 1H), 8.4 (d, 1H), 8.35 (m, 2H), 8.1 (d, 1H), 7.6 (d, 1H), 7.4 (m, 1H), 7.2 (m, 1H), 4.7 (t, 2H), 4.4 (bs, 1H), 4.0 (m, 2H), 3.4 (m, 2H), 3.8 (t, 2H), 3.2 (s, 3H), 3.1 (m, 2H), 1.8 (m, 2H), 1.7 (m, 2H). LCMS m/z 411 (M+H).

### Example 10

### [4-(5-Aminomethyl-2-fluoro-phenyl)-piperidin-1-yl]-[1-(2-methoxy-ethyl)-1H-pyrrolo[3,2-c]pyridin-3-yl]-methanone dihydrochloride

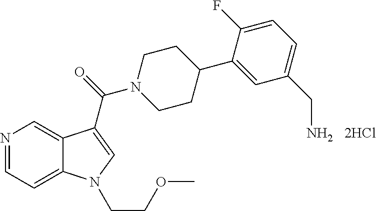

### A. 3-Iodo-1H-pyrrolo[3,2-c]pyridine

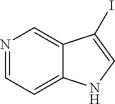

The title compound is prepared according to the procedure by Lefoix, M. et al. Synthesis 2005, 20, 3581-3588, ¹H NMR (CD₃OD, 300 MHz) δ 8.54 (s, 1H), 8.20 (d, 1H), 7.21 (s, 1H), 7.42 (d, 1H). LCMS m/z 245 (M+H).

### B. 3-Iodo-1-(2-methoxy-ethyl)-1H-pyrrolo[3,2-c]pyridine

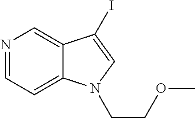

The title compound is prepared in a similar manner as Example 6A using 3-iodo-1H-pyrrolo[3,2-c]pyridine as the starting material. ¹H NMR (CDCl₃, 300 MHz) δ 8.70 (m, 1H), 8.37 (d, 1H), 7.27 (s, 1H), 7.20 (m, 1H), 4.26 (t, 2H), 3.68 (t, 2H), 3.31 (s, 3H). LCMS m/z 303 (M+H).

### C. 1-(2-Methoxy-ethyl)-1H-pyrrolo[3,2-c]pyridine-3-carboxylic acid

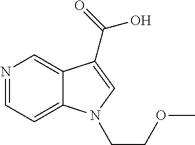

The title compound is prepared in a similar manner according to the procedure by Lefoix, M. et al. Synthesis 2005, 20, 3581-3588 using 3-iodo-1-(2-methoxy-ethyl)-1H-pyrrolo[3,2-c]pyridine as the starting material. ¹H NMR (CD₃OD, 300 MHz) δ 9.35 (s, 1H), 8.23 (d, 1H), 7.95 (s, 1H), 7.62 (d, 1H), 4.43 (t, 2H), 3.74 (t, 2H), 3.31 (s, 3H). LCMS m/z 221 (M+H).

### D. 2,2,2-Trifluoro-N-(4-fluoro-3-{1-[1-(2-methoxy-ethyl)-1H-pyrrolo[3,2-c]pyridine-3-carbonyl]-piperidin-4-yl}-benzyl)-acetamide

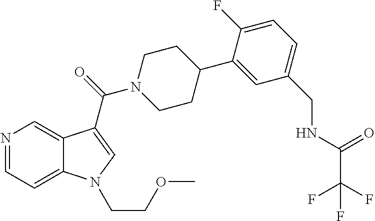

The title compound is prepared in a similar manner as Example 6C using 1-(2-methoxy-ethyl)-1H-pyrrolo[3,2-c]pyridine-3-carboxylic acid as the starting material. This material is used in the next step without further purification.

### E. [4-(5-Aminomethyl-2-fluoro-phenyl)-piperidin-1-yl]-[1-(2-methoxy-ethyl)-1H-pyrrolo[3,2-c]pyridin-3-yl]-methanone dihydrochloride

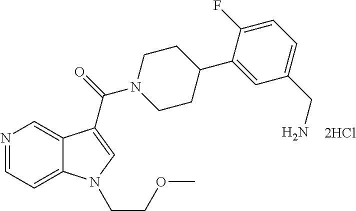

The title compound is prepared in a similar manner as Example 1D using 2,2,2-trifluoro-N-(4-fluoro-3-{1-[1-(2-methoxy-ethyl)-1H-pyrrolo[3,2-c]pyridine-3-carbonyl]-piperidin-4-yl}-benzyl)-acetamide as the starting material. ¹H NMR (DMSO-d6, 300 MHz) δ 8.44 (br s, 3H), 8.35 (s, 1H), 8.23 (br s, 1H), 7.63 (m, 1H), 7.41-7.37 (m, 1H), 7.26-7.19 (m, 1H), 6.11 (m, 1H), 6.02 (m, H), 4.62 (t, 2H), 4.45 (br s, H), 4.00 (m, 2H), 3.74 (t, 2H) 3.22 (s, 3H), 3.08-2.97 (m, 4H), 1.91-1.67 (m, 4H).). LCMS m/z 411 (M+H).

### Example 11

### [4-(5-Aminomethyl-2-fluoro-phenyl)-piperidin-1-yl]-[7-(2-methoxy-ethoxy)-1-(2-methoxy-ethyl)-1H-pyrrolo[2,3-c]pyridin-3-yl]-methanone dihydrochloride

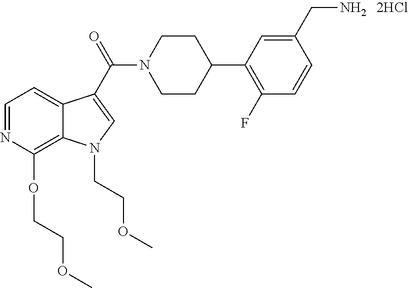

### A. 2,2,2-Trichloro-1-(7-hydroxy-1H-pyrrolo[2,3-c]pyridin-3-yl)-ethanone

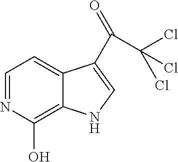

The title compound is prepared in a similar manner as Example 5A using 7-methoxy-1H-pyrrolo[2,3-c]pyridine as the starting material. ¹H NMR (DMSO-d6, 300 MHZ) δ 13.4 (bs, 1H), 11.5 (bs, 1H), 8.2 (s, 1H), 7.2 (m, 1H), 7.0 (d, 1H). LCMS m/z 279 (M+H).

### B. 7-Hydroxy-1H-pyrrolo[2,3-c]pyridine-3-carboxylic acid methyl ester

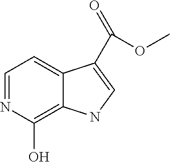

The title compound is prepared in a similar manner as Example 8A using 2,2,2-trichloro-1-(7-hydroxy-1H-pyrrolo[2,3-c]pyridin-3-yl)-ethanone as the starting material. ¹H NMR (DMSO-d6, 300 MHz) δ 12.8 (bs, 1H), 11.2 (bs, 1H), 7.8 (s, 1H), 7.0 (m, 1H), 6.8 (d, 1H), 3.8 (s, 3H). LCMS m/z 193 (M+H).

### C. 7-(2-Methoxy-ethoxy)-1-(2-methoxy-ethyl)-1H-pyrrolo[2,3-c]pyridine-3-carboxylic acid methyl ester

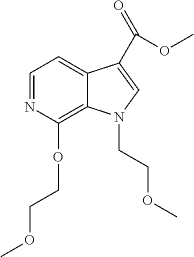

To 7-hydroxy-1H-pyrrolo[2,3-c]pyridine-3-carboxylic acid methyl ester (0.63 g, 3.28 mmol) in DMF (10 mL) under Ar is added NaH (0.26 g, 6.60 mmol). The reaction mixture is stirred for 10 min and 2-methoxyethyl bromide (1.23 mL, 13.12 mmol) is added. The reaction is stirred at room temperature overnight. The reaction mixture is poured into EtOAc and the organic layer washed with H₂O (2×) water, brine, dried with MgSO₄, filtered and concentrated in vacuo to give the crude product. Purification by flash chromatography on SiO₂ eluting with 100% EtOAc gives 0.74 g (73%) of the desired product. ¹H NMR (CDCl₃, 300 MHz) δ 7.75 (s, 1H), 7.1 (d, 1H), 7.0 (d, 1H), 4.7 (t, 2H), 4.2 (t, 2H), 3.9 (s, 3H), 3.75 (t, 2H), 3.65 (t, 2H), 3.3 (d, 6H). LCMS m/z 309 (M+H).

### D. 7-(2-Methoxy-ethoxy)-1-(2-methoxy-ethyl)-1H-pyrrolo[2,3-c]pyridine-3-carboxylic acid

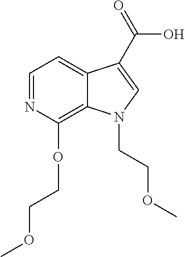

To 7-(2-methoxy-ethoxy)-1-(2-methoxy-ethyl)-1H-pyrrolo[2,3-c]pyridine-3-carboxylic acid methyl ester (0.59 g, 1.92 mmol) in MeOH (20 mL) is added 1N NaOH (20 mL). The resulting solution is stirred at room temperature overnight. The reaction mixture is acidified to pH=3 with 1N HCl and is concentrated in vacuo to remove the MeOH. The aqueous layer is lyophilized to dryness and the resulting solid is triturated with H₂O to yield 0.50 g (86%) of desired product. ¹H NMR (DMSO-d6, 300 MHz) δ 12.3 (s, 1H), 7.8 (s, 1H), 7.3 (d, 1H), 6.8 (d, 1H), 4.6 (t, 2H), 4.1 (t, 2H), 3.7 (t, 2H), 3.6 (t, 2H), 3.2 (d, 6H). LCMS m/z 295 (M+H).

### E. 2,2,2-Trifluoro-N-(4-fluoro-3-{1-[7-(2-methoxy-ethoxy)-1-(2-methoxy-ethyl)-1H-pyrrolo[2,3-c]pyridine-3-carbonyl]-piperidin-4-yl}-benzyl)-acetamide

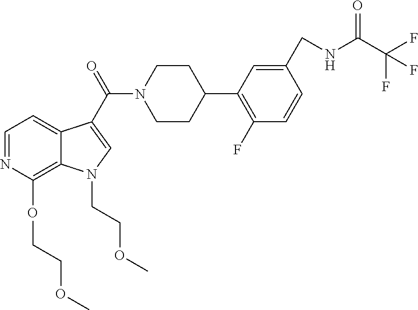

The title compound is prepared in a similar manner as Example 1C using 7-(2-methoxy-ethoxy)-1-(2-methoxy-ethyl)-1H-pyrrolo[2,3-c]pyridine-3-carboxylic acid as the starting material. ¹H NMR (CDCl₃, 300 MHz) δ 7.4 (s, 1H), 7.15 (m, 2H), 7.05 (m, 2H), 6.65 (d, 1H), 6.55 (bs, 1H), 4.75 (t, 2H), 4.6 (bs, 1H), 4.45 (m, 3H), 4.2 (t, 2H), 3.75 (t, 2H), 3.7 (t, 2H), 3.3 (s, 6H), 3.1 (m, 3H), 1.9 (m, 2H), 1.8 (m, 2H). LCMS m/z 581 (M+H).

### F. [4-(5-Aminomethyl-2-fluoro-phenyl)-piperidin-1-yl]-[7-(2-methoxy-ethoxy)-1-(2-methoxy-ethyl)-1H-pyrrolo[2,3-c]pyridin-3-yl]-methanone hydrochloride

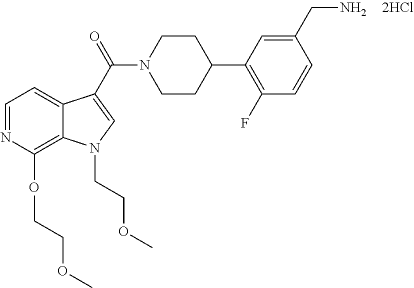

The title compound is prepared in a similar manner as Example 5D using 2,2,2-trifluoro-N-(4-fluoro-3-{1-[7-(2-methoxy-ethoxy)-1-(2-methoxy-ethyl)-1H-pyrrolo[2,3-c]pyridine-3-carbonyl]-piperidin-4-yl}-benzyl)-acetamide as the starting material. ¹H NMR (DMSO-d6, 300 MHz) δ 8.2 (bs, 2H), 7.7 (s, 1H), 7.5 (m, 1H), 7.35 (m, 1H), 7.2 (m, 2H), 7.1 (m, 1H), 6.5 (d, 1H), 4.7 (t, 2H), 4.4 (bs, 2H), 4.15 (t, 2H), 4.0 (m, 2H), 3.7 (t, 2H), 3.6 (t, 2H), 3.2 (d, 6H), 3.1 (m, 3H), 1.8 (m, 2H), 1.7 (m, 2H). LCMS m/z 485 (M+H).

### Example 12

### [4-(5-Aminomethyl-2-fluoro-phenyl)-piperidin-1-yl]-[1-(2-methoxy-ethyl)-7-methyl-1H-pyrrolo[3,2-b]pyridin-3-yl]-methanone dihydrochloride

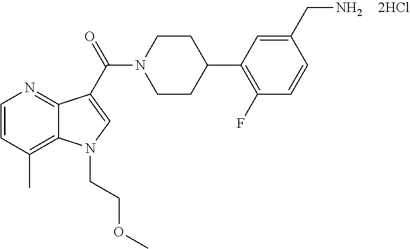

### A. 7-Methyl-1H-pyrrolo[3,2-b]pyridine

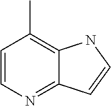

The title compound is prepared according to the following procedure: Journal of Organic Chemistry 2002, 67(7), 2345-2347. ¹H NMR (DMSO-d6, 300 MHz) δ 11.4 (bs, 1H), 8.2 (d, 1H), 7.6 (d, 1H), 6.9 (d, 1H), 6.5 (d, 1H), 3.3 (s, 3H). LCMS m/z 133 (M+H).

### B. 3-Iodo-7-methyl-1H-pyrrolo[3,2-b]pyridine

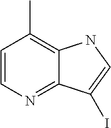

To a solution of 7-methyl-1H-pyrrolo[3,2-b]pyridine (0.50 g, 3.79 mmol) in THF (30 mL) is added N-iodosuccinimide (0.34 g, 4.2 mmol). The reaction mixture is stirred for 2 h and is concentrated in vacuo. Purification by flash chromatography on SiO₂ eluting with 50% EtOAc/heptane gives 0.92 g (94%) of the desired product. ¹H NMR (DMSO-d6, 300 MHz) δ 11.9 (bs, 1H), 8.2 (d, 1H), 7.8 (s, 1H), 7.0 (d, 1H), 6.5 (d, 1H), 3.3 (s, 3H). LCMS m/z 259 (M+H).

### C. 3-Iodo-1-(2-methoxy-ethyl)-7-methyl-1H-pyrrolo[3,2-b]pyridine

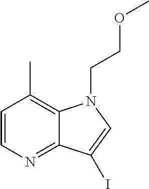

A mixture of powder KOH (1.74 g, 31 mmol) in DMSO (60 mL) is stirred at room temperature for 10 min. 3-Iodo-7-methyl-1H-pyrrolo[3,2-b]pyridine (2.00 g, 7.75 mmol) is added. The reaction mixture is stirred for 1 h and then 2-methoxyethyl bromide (1.46 mL, 15.5 mmol) is added. After 3 h the reaction mixture is poured into EtOAc and the organic layer washed with H₂O (2×), brine, dried with MgSO₄, filtered and concentrated in vacuo. Purification by flash chromatography on SiO₂ eluting with 50% EtOAc/heptane gives 1.93 g (79%) of the desired product. ¹H NMR (CDCl₃, 300 MHz) δ 8.4 (d, 1H), 7.4 (s, 1H), 6.9 (d, 1H), 4.45 (t, 2H), 3.65 (t, 2H), 3.3 (s, 3H), 2.7 (s, 3H). LCMS m/z 317 (M+H).

### D. 1-(2-Methoxy-ethyl)-7-methyl-1H-pyrrolo[3,2-b]pyridine-3-carboxylic acid trifluoro-acetic acid salt

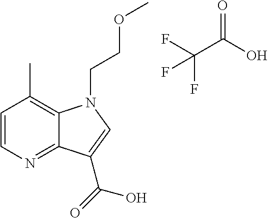

The title compound is prepared in a similar manner as Example 10C using 3-iodo-1-(2-methoxy-ethyl)-7-methyl-1H-pyrrolo[3,2-b]pyridine as the starting material. ¹H NMR (DMSO-d6) δ 8.7 (s, 1H), 8.5 (d, 1H), 7.6 (d, 1H), 4.75 (t, 2H), 3.75 (t, 2H), 3.2 (s, 3H), 2.95 (s, 3H). LCMS m/z 235 (M+H).

### E. 2,2,2-Trifluoro-N-(4-fluoro-3-{1-[1-(2-methoxy-ethyl)-7-methyl-1H-pyrrolo[3,2-b]pyridine-3-carbonyl]-piperidin-4-yl}-benzyl)-acetamide

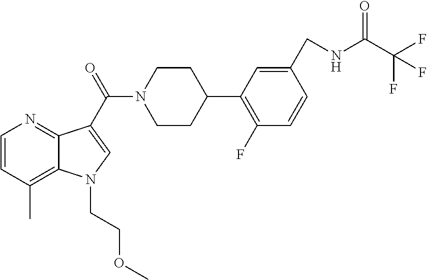

The title compound is prepared in a similar manner as Example 1C using 1-(2-methoxy-ethyl)-7-methyl-1H-pyrrolo[3,2-b]pyridine-3-carboxylic acid trifluoroacetic acid salt as the starting material. ¹H NMR (CDCl₃, 300 MHz) δ 8.4 (d, 1H), 7.8 (s, 1H), 7.25-7.1 (m, 2H), 7.0 (m, 1H), 6.9 (d, 1H), 6.7 (bs, H), 4.5 (m, 4H), 3.75 (t, 2H), 3.3 (s, 3H), 3.15 (m, 2H), 2.7 (s, 3H), 1.9-1.75 (m, 4H), 1.6 (m, 3H). LCMS m/z 521 (M+H).

### D. [4-(5-Aminomethyl-2-fluoro-phenyl)-piperidin-1-yl]-[1-(2-methoxy-ethyl)-7-methyl-1H-pyrrolo[3,2-b]pyridin-3-yl]-methanone dihydrochloride

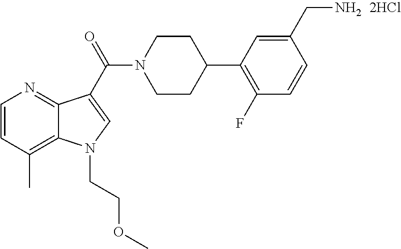

The title compound is prepared in a similar manner as Example 5D using 2,2,2-trifluoro-N-(4-fluoro-3-{1-[1-(2-methoxy-ethyl)-7-methyl-1H-pyrrolo[3,2-b]pyridine-3-carbonyl]-piperidin-4-yl}-benzyl)-acetamide as the starting material. ¹H NMR (DMSO-d6, 300 MHz) δ 8.6 (m, 2H), 8.4 (bs, 2H), 7.6 (m, 2H), 7.4 (m, 1H), 7.2 (m, 1H), 4.7 (t, 2H), 4.0 (m, 2H), 3.7 (t, 2H), 3.6-3.3 (m, 4H), 3.2 (m, 4H), 3.0 (s, 3H), 2.0-1.8 (m, 4H). LCMS m/z 425 (M+H).

### Example 13

### [4-(5-Aminomethyl-2-fluoro-phenyl)-piperidin-1-yl]-[7-chloro-1-(2-methoxy-ethyl)-1H-pyrrolo[2,3-c]pyridin-3-yl]-methanone dihydrochloride

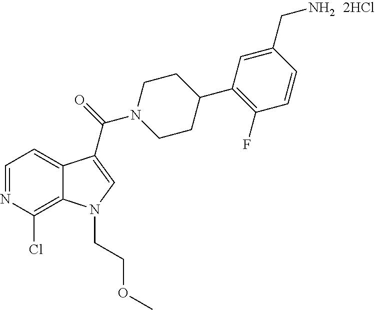

### A. 7-Chloro-1H-pyrrolo[2,3-c]pyridine

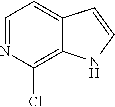

To a 1 L three-necked flask under nitrogen is added commercially available vinylmagnesium bromide solution (1 M in THF, 500 mL, 500 mmol). At 0° C. a solution of 2-chloro-3-nitro-pyridine (25 g, 160 mmol) in THF (100 mL) is added dropwise via addition funnel over 40 minutes. After stirring an additional 40 minutes at 0° C. the reaction is quenched with aqueous saturated NH₄Cl solution and extracted with EtOAc. The combined organic layers are dried over MgSO₄, filtered and concentrated in vacuo. The crude material is passed through a plug of silica gel with CH₂Cl₂/heptanes (33%) as eluant to give a solid that is recrystallized from CH₂Cl₂/heptanes to deliver 6.9 g (28%) of the titled product as a beige solid mp 182-185° C. ¹H NMR (CDCl₃) δ 8.60 (br s, 1H), 8.05 (m, 1H), 7.50 (m, 1H), 7.42 (m, 1H), 6.64 (m, 1H). LCMS m/z: 153 (M+H).

### B. 7-Chloro-1-(2-methoxy-ethyl)-1H-pyrrolo[2,3-c]pyridine

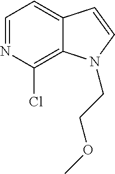

To a 0° C. solution of 7-chloro-1H-pyrrolo[2,3-c]pyridine (1.3 g, 8.5 mmol) in DMF (40 mL) under nitrogen is added sodium hydride (60% suspension in oil, 0.51 g, 12.8 mmol). After stirring for 10 minutes at 0° C., 1-bromo-2-methoxy-ethane (1.8 g, 12.8 mmol) is added followed by a catalytic amount of NaI. After stirring an additional 2 h at 0° C. the reaction is quenched with aqueous saturated NaHCO₃ solution and extracted with EtOAc. The combined organic layers are dried over MgSO₄, filtered and concentrated in vacuo. The crude material is purified on silica gel with 30% EtOAc/heptanes as eluant to deliver the titled compound as a clear colorless oil 1.66 g (93%). ¹H NMR (CDCl₃) δ 7.98 (d, J=5.4 Hz, 1H), 7.45 (d, J=5.4 Hz, 1H), 7.31 (d, J=3.1 Hz, 1H), 6.52 (d, J=3.1 Hz, 1H), 4.72 (t, J=5.1 Hz, 2H), 3.76 (t, J=5.1 Hz, 2H), 3.30 (s, 3H). LCMS m/z 211 (M+H).

### C. 1-[7-Chloro-1-(2-methoxy-ethyl)-1H-pyrrolo[2,3-c]pyridin-3-yl]-2,2,2-trifluoro-ethanone

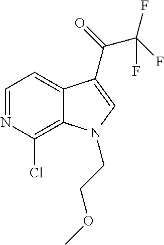

To a 0 C solution of 7-chloro-1-(2-methoxy-ethyl)-1H-pyrrolo[2,3-c]pyridine (1.5 g, 7.1 mmol) in DMF (10 mL) under nitrogen is added trifluoracetic anhydride (4.5 g, 21.3 mmol). After stirring for 2 h at 0° C., an additional trifluoracetic anhydride (4.5 g, 21.3 mmol) is added and the reaction allowed to warm to rt overnight. At 0° C. the reaction is quenched with aqueous saturated NaHCO₃ solution and extracted with EtOAc. The combined organic layers are dried over MgSO₄, filtered and concentrated in vacuo. The crude material is recrystallized from CH₂Cl₂/heptanes to deliver 1.75 g (80%) as a first crop of the titled product as a beige solid mp 110-112° C. ¹H NMR (CDCl₃) δ 8.27 (s, 2H), 8.09 (s, 1H), 4.82 (t, J=5.1 Hz, 2H), 3.81 (t, J=5.1 Hz, 2H), 3.32 (s, 3H). LCMS m/z 307 M+H).

### D. 7-Chloro-1-(2-methoxy-ethyl)-1H-pyrrolo[2,3-c]pyridine-3-carboxylic acid

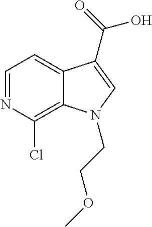

To a mixture of 1-[7-chloro-1-(2-methoxy-ethyl)-1H-pyrrolo[2,3-c]pyridin-3-yl]-2,2,2-trifluoro-ethanone (1.0 g, 3.27 mmol) in THF (5 mL) and H₂O (25 mL) is added lithium hydroxide (1.4 g, 32.7 mmol). After refluxing the mixture for 1.5 h the reaction is cooled to rt and extracted with Et₂O. The aqueous later is acidified with aqueous 10% HCl and extracted with hot EtOAc. The combined organic layers are dried over MgSO₄, filtered and concentrated in vacuo. The crude material is recrystallized from EtOAc/MeOH to deliver 0.76 g (91%) of the titled compound as a white powder mp 210-203° C. ¹H NMR (DMSO-d6) δ 12.55 (s, 1H), 8.28 (s, 1H), 8.07 (m, 1H), 7.97 (m, 1H), 4.78 (t, J=5.1 Hz, 2H), 3.73 (t, J=5.1 Hz, 2H), 3.32 (s, 3H). LCMS m/z 255 (M+H).

### E. N-(3-{1-[7-Chloro-1-(2-methoxy-ethyl)-1H-pyrrolo[2,3-c]pyridine-3-carbonyl]-piperidin-4-yl}-4-fluoro-benzyl)-2,2,2-trifluoro-acetamide

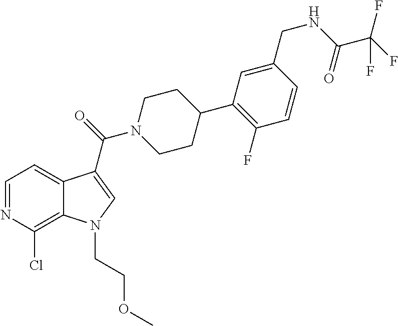

To a mixture of 7-chloro-1-(2-methoxy-ethyl)-1H-pyrrolo[2,3-c]pyridine-3-carboxylic acid (0.22 g, 0.86 mmol) in THF (10 mL) under nitrogen is added carbonyl diimidazole (0.17, 1.04 mmol). After stirring 6 h at ambient temperature 2,2,2-trifluoro-N-(4-fluoro-3-piperidin-4-yl-benzyl)-acetamide (0.52 g, 1.73 mmol) is added and the reaction is stirred overnight. The reaction is quenched with aqueous 10% HCl solution and extracted with EtOAc. The combined organic layers are washed with aqueous saturated NaHCO₃ solution, dried over MgSO₄, filtered and concentrated in vacuo to deliver 0.36 g (77%) of the titled compound as a white foam. ¹H NMR (CDCl₃) δ 8.10 (m, 1H), 7.66 (m, 1H), 7.61 (m, 1H), 7.10 (m, 4H), 6.50 (br s, 1H), 4.76 (m, 2H), 4.51 (m, 5H), 3.78 (m, 2H), 3.31 (s, 3H), 3.16 (m, 1H), 1.92 (m, 1H) 1.75 (m, 1H). LCMS m/z 541 (M+H).

### F. [4-(5-Aminomethyl-2-fluoro-phenyl)-piperidin-1-yl]-[7-chloro-1-(2-methoxy-ethyl)-1H-pyrrolo[2,3-c]pyridin-3-yl]-methanone dihydrochloride

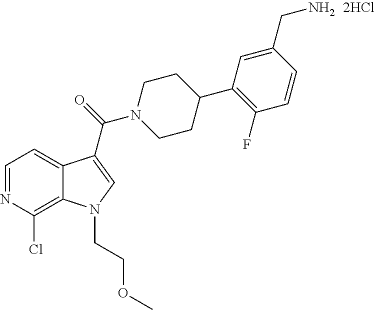

To a mixture of N-(3-{1-[7-chloro-1-(2-methoxy-ethyl)-1H-pyrrolo[2,3-c]pyridine-3-carbonyl]-piperidin-4-yl}-4-fluoro-benzyl)-2,2,2-trifluoro-acetamide (0.5 g, 0.92 mmol) in (30 mL) and H₂O (15 mL) is added of Na₂CO₃ (0.98 g, 9.2 mmol). After heating 40 min on a steam bath the mixture is concentrated in vacuo and extracted with EtOAc/H₂O. The combined organic layers are dried over MgSO₄, filtered and concentrated in vacuo. The crude material is passed through a plug of silica gel (EtOAc eluant) to remove baseline material. This material is treated with methanolic HCl. This mixture is concentrated in vacuo. The resulting hydrochloride salt is recrystallized from MeOH/EtOAc to deliver 0.25 g (57%) of the titled compound as a white powder mp 163-167° C. ¹H NMR (DMSO-d6) δ 8.23 (m, 3H), 8.04 (m, 1H), 7.70 (m, 1H), 7.56 (m, 1H), 7.38 (m, 1H), 7.26 (m, 1H), 4.78 (m, 2H), 4.40-4.00 (m, 7H), 3.99 (m, 2H), 3.23 (s, 3H), 3.17 (m, 2H), 1.83 (m, 1H) 1.72 (m, 1H). LCMS m/z 445 (M+H).

### Example 14

### [4-(5-Aminomethyl-2-fluoro-phenyl)-piperidin-1-yl]-[1-(2-methoxy-ethyl)-7-methyl-1H-pyrrolo[2,3-c]pyridin-3-yl]-methanone

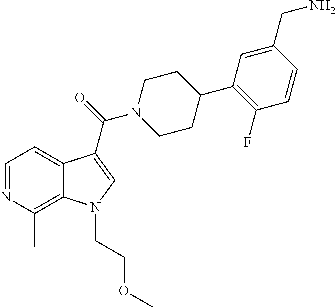

### A. 2,2,2-Trifluoro-1-[1-(2-methoxy-ethyl)-7-methyl-1H-pyrrolo[2,3-c]pyridin-3-yl]-ethanone

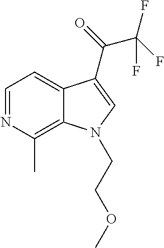

To a mixture of 1-[7-chloro-1-(2-methoxy-ethyl)-1H-pyrrolo[2,3-c]pyridin-3-yl]-2,2,2-trifluoro-ethanone (1.40 g, 4.57 mmol) in dioxane (40 mL) under nitrogen is added methylboronic acid (0.83, 13.7 mmol), K₂CO₃ (1.90 g, 13.7 mmol) and tetrakis(triphenylphosphine)palladium (0) (0.21 g, 0.18 mmol). After refluxing the reaction 5 h silica gel is added and the mixture concentrated in vacuo. The resulting powder was added to the top of a silica plug and eluted with EtOAc. Appropriate fractions are concentrated to give a solid which is recrystallized from CH₂Cl₂/heptanes to deliver 1.20 g (92%) of the titled product as a beige solid mp 125-127° C.) ¹H NMR (CDCl₃) δ 8.39 (m, 1H), 8.18 (m, 1H), 8.05 (m, 1H), 4.64 (t, J=5.1 Hz, 2H), 3.77 (t, J=5.1 Hz, 2H), 3.33 (s, 3H), 2.94 (s, 3H). LCMS m/z 287 (M+H).

### B. 1-(2-Methoxy-ethyl)-7-methyl-1H-pyrrolo[2,3-c]pyridine-3-carboxylic acid

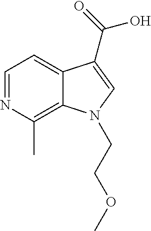

A suspension of 2,2,2-trifluoro-1-[1-(2-methoxy-ethyl)-7-methyl-1H-pyrrolo[2,3-c]pyridin-3-yl]-ethanone (0.70 g, 2.40 mmol) in 6 N NaOH solution (6 mL) is heated to reflux for 15 minutes. The resulting clear solution is cooled to 0° C. and acidified with 10% aqueous HCl. This mixture is concentrated in vacuo. The resulting crude product is passed through a silica gel plug (3% AcOH/20% MeOH/CH₂Cl₂ as eluant). Concentration in vacuo delivers 0.33 g (59%) of the titled compound as a white powder mp 215-225° C. ¹H NMR (DMSO-d6) δ 8.02 (m, 1H), 7.92 (m, 1H), 7.88 (m, 1H), 4.60 (t, J=5.3 Hz, 2H), 3.68 (t, J=5.3 Hz, 2H), 3.22 (s, 3H), 1.88 (s, 3H). LCMS m/z 235 (M+H).

### C. 2,2,2-Trifluoro-N-(4-fluoro-3-{1-[1-(2-methoxy-ethyl)-7-methyl-1H-pyrrolo[2,3-c]pyridine-3-carbonyl]-piperidin-4-yl}-benzyl)-acetamide

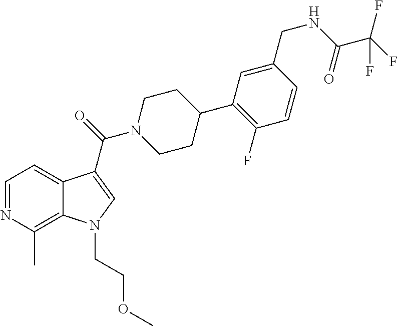

To a mixture of 1-(2-methoxy-ethyl)-7-methyl-1H-pyrrolo[2,3-c]pyridine-3-carboxylic acid (0.50 g, 2.1 mmol) in THF (15 mL0 under nitrogen is added carbonyl diimidazole (0.69 g, 4.27 mmol). After stirring 1 h at ambient temperature 2,2,2-trifluoro-N-(4-fluoro-3-piperidin-4-yl-benzyl)-acetamide (2.6 g, 8.5 mmol) is added and the reaction is heated to reflux overnight. The reaction is quenched with saturated aqueous NH₄Cl solution and extracted with EtOAc. The combined organic layers are dried over MgSO₄, filtered and concentrated in vacuo. The crude product is purified on silica gel (2.5% MeOH/CH₂Cl₂ eluant) to deliver 0.36 g (77%) of the titled compound as a white foam. ¹H NMR (CDCl₃) δ 8.21 (m, 1H), 7.52 (m, 1H), 7.2-7.0 (m, 4H), 6.62 (br s, 1H), 4.58 (m, 2H), 4.49 (m, 6H), 3.73 (m, 2H), 3.31 (s, 3H), 3.10 (m, 2H), 2.93 (s, 3H), 1.9-1.6 (m, 3H). LCMS m/z 521 (M+H).

### D. [4-(5-Aminomethyl-2-fluoro-phenyl)-piperidin-1-yl]-[1-(2-methoxy-ethyl)-7-methyl-1H-pyrrolo[2,3-c]pyridin-3-yl]-methanone

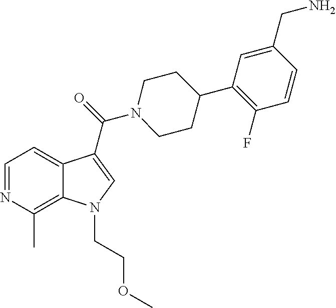

To a mixture of 2,2,2-trifluoro-N-(4-fluoro-3-{1-[1-(2-methoxy-ethyl)-7-methyl-1H-pyrrolo[2,3-c]pyridine-3-carbonyl]-piperidin-4-yl}-benzyl)-acetamide (0.42 g, 0.81 mmol) in MeOH (8 mL) and H₂O (2 mL) is added of aqueous 50% NaOH solution (1.0 mL). After stirring at ambient temperature for 1 h the mixture is concentrated in vacuo, diluted with MeOH and adsorbed onto silica gel. This material is purified on silica gel (CH₂Cl₂, 5% MeOH/CH₂CL₂ and finally 5% 7N NH₃/MeOH: 95% CH₂Cl₂ as eluant). Concentration of appropriate fractions delivers 0.25 g (73%) of the titled compound as a white foam. ¹H NMR (CDCl₃) δ 8.22 (m, 1H), 7.68 (m, 1H), 7.59 (m, 2H), 7.26-6.96 (m, 4H), 4.58 (m, 3H), 3.85 (m, 2H), 3.73 (m, 2H), 3.31 (s, 3H), 3.10 (m, 3H), 2.93 (s, 3H), 1.90-1.60 (m, 5H). LCMS m/z 425 (M+H).

### Example 15

### [4-(5-Aminomethyl-2-fluoro-phenyl)-piperidin-1-yl]-[7-methoxy-1-(2-methoxy-ethyl)-1H-pyrrolo[3,2-b]pyridin-3-yl]-methanone dihydrochloride

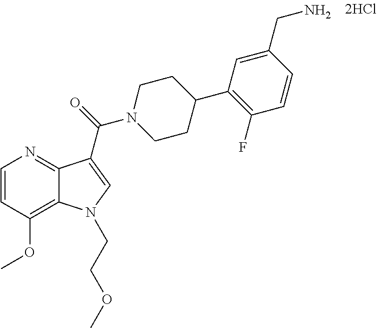

### A. 4-Methoxy-3-nitro-pyridine

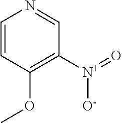

To conc. H₂SO₄ (5 mL) chilled in an ice bath is added 4-methoxypyridine (0.5 mL, 4.9 mmol) dropwise over a 20 s period. Conc. Fuming nitric acid (5 mL) is added, and the reaction mixture is heated at 70° C. for 2.5 days. This mixture is cooled to rt, and then is poured into ice. Soild K₂CO₃ is added until the pH of the mixture is basic. The mixture is partitioned between H₂O and EtOAc. The two layers is separated, and the aqueous layer is extracted with EtOAc once. The combined organic layers are washed with H₂O and brine, dried over MgSO₄, filtered, and concentrated in vacuo to yield 0.7 g (92%) of the product a yellow powder. ¹H NMR (CDCl₃, 300 MHz) δ 9.02 (s, 1H), 8.65 (d, J=5.8, 1H), 7.04 (d, J=5.9, 1H), 4.05 (s, 3H). LC Rt: 0.5 min; LCMS m/z 155 (M+1, 100%).

### B. 4-Methoxy-pyridin-3-ylamine

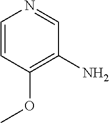

A mixture of 4-methoxy-3-nitro-pyridine (19.2 g, 0.13 mol) and Pd/C (10%, 1.5 g) in MeOH (150 mL) is hydrogenated at 40 psi for 5 h or until no more H₂ is consumed. The mixture is filtered through Celite, and the filtrate is concentrated in vacuo. The residue is dissolved in CH₂Cl₂, and the resulting solution is dried over MgSO₄, filtered, and concentrated in vacuo to yield 15.0 g of the product as a yellow liquid. ¹H NMR (CDCl₃, 300 MHz) δ 8.00 (s, 1H), 7.98 (d, J=5.5, 1H), 6.70 (d, J=5.4, 1H) 3.90 (s, 3H), 3.71 (br s, 2H). LC Rt: 0.57 min; LCMS m/z 125 (M+1, 100%).

### C. 2-Bromo-4-methoxy-pyridin-3-ylamine

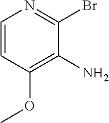

To a solution of 4-methoxy-pyridin-3-ylamine (6.76 g, 54.5 mmol) in conc. HCl (50 mL) is added Br₂ (3.36 mL, 65.4 mmol) dropwise over a 30 s period. This mixture is stirred at rt for 1 h, and then heated at 55° C. overnight. The mixture is cooled to rt, and then poured into ice. Conc. NH₄OH is added until the pH of the solution is basic. The resulting suspension is partitioned between H₂O and EtOAc. The two layers are separated, and the aqueous layer is extracted with EtOAc (2×). The combined organic layers are washed with H₂O and brine, dried over MgSO₄, filtered, and concentrated in vacuo. The crude material is purified on silica gel with EtOAc/MeOH (100% to 80/20) as elant to yield 9.18 g (82%) of the product as a white powder. ¹H NMR (CDCl₃, 300 MHz) 7.75 (d, J=5.3, 1H), 6.68 (d, J=5.3, 1H), 4.11 (br s, 2H), 3.91 (s, 3H). LC Rt: 0.89 min; LCMS m/z 203 (M+1, 100%).

### D. (2-Bromo-4-methoxy-pyridin-3-yl)-carbamic acid ethyl ester

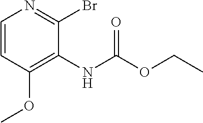

To a solution of 2-bromo-4-methoxy-pyridin-3-ylamine (540 mg, 2.66 mmol) in pyridine (20 mL) at 0° C. is added ethyl chloroformate (0.38 mL, 3.99 mmol). After 30 min, more chloroformate is added (˜18 mmol) is added until the reaction goes to completion. The mixture is partitioned between sat. NaHCO₃ and EtOAc. The two layers are separated, and the aqueous layer is extracted with EtOAc once. The combined organic layers are washed with H₂O and brine, dried over MgSO₄, filtered, and concentrated in vacuo. The crude material is purified on silica gel with EtOAc/MeOH (100/0 to 90/10) as eluant to yield 0.54 g of the product as a white crystalline solid. ¹H NMR (CDCl₃, 300 MHz) 8.18 (d, J=5.6, 1H), 6.84 (d, J=5.7, 1H), 6.02 (br s, 1H), 4.23 (q, J=7.0, 2H), 3.92 (s, 3H), 1.31 (t, J=7.2, 3H). LC Rt: 1.89 min; LCMS m/z 275 (M+1, 100%).

### E. (4-Methoxy-2-trimethylsilanylethynyl-pyridin-3-yl)-carbamic acid ethyl ester

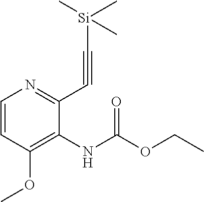

A mixture of (2-bromo-4-methoxy-pyridin-3-yl)-carbamic acid ethyl ester (540 mg, 1.96 mmol), Et₃N (0.54 mL, 3.9 mmol), Pd(PPh)₂Cl₂ (69 mg, 5% mol), CuI (30 mg, 8% mol), and TMS-acetylene (0.56 mL, 3.9 mmol) in degassed THF (10 mL) is heated at 60° C. overnight. The mixture is cooled to rt, and then partitioned between H₂O and EtOAc. This mixture is filtered through Celite to remove the insoluble material. The two layers of the filtrate are separated, and the organic layer is washed H₂O and brine, dried over MgSO₄, filtered, and concentrated in vacuo. The crude material is purified on silica gel with heptane/EtOAc (50/50 to 0/100) as eluant to give 460 mg (80%) of the product as a beige solid. ¹H NMR (CDCl₃, 300 MHz) δ 8.32 (d, J=5.6, 1H), 6.81 (d, J=5.7, 1H), 6.16 (br s, 1H), 4.22 (q, J=7.0, 2H), 3.90 (s, 3H), 1.30 (t, J=7.2, 3H), 0.26 (s, 9H). LC Rt 2.63 min; LCMS m/z 293 (M+1, 100%).

### F. 7-Methoxy-1H-pyrrolo[3,2-b]pyridine

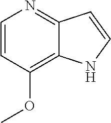

A mixture of (4-methoxy-2-trimethylsilanylethynyl-pyridin-3-yl)-carbamic acid ethyl ester (460 mg, 1.57 mmol) and KOH (353 mg, 6.29 mmol) in t-BuOH (20 mL) is heated at 60° C. for 6 h, and then stirred at rt overnight. The mixture is concentrated in vacuo. The residue is partitioned between H₂O and EtOAc. The two layers are separated, and the organic layer is washed with brine, dried over MgSO₄, filtered, and concentrated in vacuo. The crude material is purified on silica gel with EtOAc/MeOH (90/10 to 80*20) as eluant to yield 127 mg (54%) of the product as a white powder. ¹H NMR (CD₃OD, 300 MHz) δ 8.17 (d, J=5.5, 1H), 7.42 (d, J=3.2, 1H), 6.75 (d, J=5.5, 1H), 6.53 (d, J=3.2, 1H), 4.85 (s, 3H). LC Rt 0.39 min; LCMS m/z 149 (M+1, 100%).

### G. 7-Methoxy-1-(2-methoxy-ethyl)-1H-pyrrolo[3,2-b]pyridine

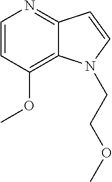

The title compound is prepared in a similar manner as Example 12C using 7-methoxy-1H-pyrrolo[3,2-b]pyridine as the starting material. ¹H NMR (CDCl₃, 300 MHz) δ 8.29 (d, J=5.5, 1H), 7.23 (d, J=3.1, 1H), 6.61 (d, J=3.1, 1H), 6.56 (d, J=5.5, 1H), 4.50 (t, J=5.7, 2H), 3.97 (s, 3H), 3.68 (t, J=5.5, 2H), 3.29 (s, 3H). LC Rt 2.51 min; MS m/z 207 (M+1, 100%).

### H. 3-Iodo-7-methoxy-1-(2-methoxy-ethyl)-1H-pyrrolo[3,2-b]pyridine

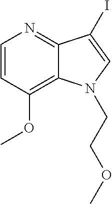

A mixture of 7-methoxy-1-(2-methoxy-ethyl)-1H-pyrrolo[3,2-b]pyridine (256 mg, 1.24 mmol) and N-iodosuccimide (536 mg, 1.61 mmol) in degassed THF (20 mL) is stirred at 60° C. for 1.5 h. The mixture is cooled to rt, and then partitioned between 0.1 M NaOH and EtOAc. The two layers are separated, and the organic layer is washed with H₂O and brine, dried over MgSO₄, filtered, and concentrated in vacuo. The crude material is purified on silica gel with heptane/EtOAc (40/60 to 0/100) as eluant to yield 272 mg (66%) of the product as a yellow gum. ¹H NMR (CDCl₃, 300 MHz) δ 8.41 (d, J=5.5, 1H), 7.35 (s, 1H), 6.63 (d, J=5.5, 1H), 4.51 (t, J=5.3, 2H), 3.99 (s, 3H), 3.67 (t, J=5.3, 2H), 3.30 (s, 3H); LC Rt 1.43 min; MS m/z 333 (M+1, 100%).

### I. 7-Methoxy-1-(2-methoxy-ethyl)-1H-pyrrolo[3,2-b]pyridine-3-carboxylic acid ethyl ester

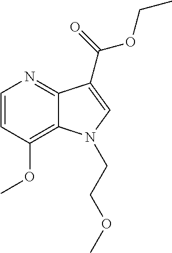

To a solution of 3-iodo-7-methoxy-1-(2-methoxy-ethyl)-1H-pyrrolo[3,2-b]pyridine (272 mg, 0.82 mmol) in THF (5 mL) at −78° C. is added n-BuLi (2.0 M in pentane, 0.62 mL, 1.24 mmol). After 15 min, diethyl carbonate (0.30 mL, 2.45 mmol) is added. After 30 min, sat. NH₄Cl is added, and the mixture is partitioned between H₂O and EtOAc. The two layers are separated, and the aqueous is extracted with EtOAc once. The combined organic layers are washed with H₂O and brine, dried over MgSO4, filtered, and concentrated in vacuo. The crude material is purified on silica gel with EtOAc/MeOH (100/0 to 80/20) as eluant to give 108 mg (47%) of the product (contaminated with ˜20% of the de-iodinated starting material) as a clear colorless film. This material is used in the next sep without further purification. ¹H NMR (CDCl₃, 300 MHz) δ 8.51 (d, J=5.4, 1H), 7.91 (s, 1H), 6.64 (d, J=5.5, 1H), 4.55-4.35 (m, 4H), (t, J=5.3, 2H), 3.99 (s, 3H), 3.70 (t, J=5.2, 2H), 3.29 (s, 3H), 1.41 (t, J=7.1, 3H). LC Rt 0.50 min; MS m/z 279 (M+1, 100%).

### J. 7-Methoxy-1-(2-methoxy-ethyl)-1H-pyrrolo[3,2-b]pyridine-3-carboxylic acid hydrochloride

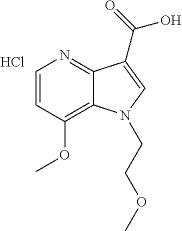

A mixture of 7-methoxy-1-(2-methoxy-ethyl)-1H-pyrrolo[3,2-b]pyridine-3-carboxylic acid ethyl ester (108 mg, 0.39 mmol) in MeOH (5 mL) and NaOH (1.0 M, 2 mL) is heated at 45° C. overnight and then at 60° C. for 3 h. The mixture is cooled to rt, and then partitioned between H₂O and EtOAc. The two layers are separated, and the aqueous is extracted with EtOAc once. The aqueous layer is acidified to pH ˜2 with 3 M HCl. This acidified solution is concentrated to dryness. The residue is suspended in toluene and then concentrated to dryness. The resulting white solid was dried in vacuo for 1 h, and then is use in the next step with further purification. LC Rt 0.31 min; MS m/z 251 (M+1, 100%).

### K. 2,2,2-Trifluoro-N-(4-fluoro-3-{1-[7-methoxy-1-(2-methoxy-ethyl)-1H-pyrrolo[3,2-b]pyridine-3-carbonyl]-piperidin-4-yl}-benzyl)-acetamide

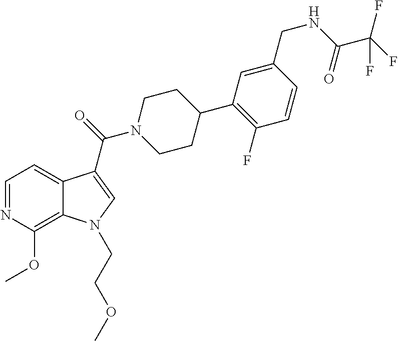

The title compound is prepared in a similar manner as Example 1C using 7-methoxy-1-(2-methoxy-ethyl)-1H-pyrrolo[3,2-b]pyridine-3-carboxylic acid hydrochloride as the starting material. ¹H NMR (CDCl₃, 300 MHz) δ 8.37 (d, J=5.4, 1H), 7.68 (s, 1H), 7.25-7.05 (m, 2H), 7.05-6.95 (m, 1H), 6.71 (br s, 1H), 6.62 (d, J=5.4, 1H), 4.70 (br s, 1H), 4.60-4.30 (m, 4H), 3.99 (s, 3H), 3.72 (t, J=5.5, 2H), 3.33 (s, 3H), 3.30-2.85 (m, 4H), 2.00-1.70 (m, 4H); ¹⁹F NMR (CDCl₃, 282 MHz) δ−75.29 (s, 3F), −119.18 (s, 1F). LC Rt 2.47 min; MS m/z 537 (M+1, 100%).

### L. [4-(5-Aminomethyl-2-fluoro-phenyl)-piperidin-1-yl]-[7-methoxy-1-(2-methoxy-ethyl)-1H-pyrrolo[3,2-b]pyridin-3-yl]-methanone dihydrochloride

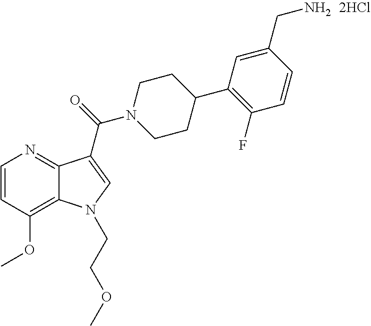

The title compound is prepared in a similar manner as Example 1D using 2,2,2-trifluoro-N-(4-fluoro-3-{1-[7-methoxy-1-(2-methoxy-ethyl)-1H-pyrrolo[3,2-b]pyridine-3-carbonyl]-piperidin-4-yl}-benzyl)-acetamide as the starting material. ¹H NMR (DMSO-d6, 300 MHz) δ 8.53 (d, J=6.5, 1H), 8.45 (br, s 3H), 8.34 (s, 1H), 7.61 (d, J=6.4, 1H), 7.50-7.30 (m, 3H), 7.30-7.10 (m, 1H), 4.80-4.55 (m, 2H), 4.50 (br m, 1H), 4.42 (s, 3H), 4.10-3.90 (m, 2H), 3.90-3.60 (m, 3H), 3.55-3.40 (m, 1H), 3.23 (s, 3H), 3.20-3.10 (m, 2H), 2.00-1.60 (m, 4H); ¹⁹F NMR (DMSO-d6, 282 MHz) −119.58 (s, 1F). LC 1.39 min; MS m/z 441 (M+1), 233 (100%).

### Example 16

### [4-(5-Aminomethyl-2-fluoro-phenyl)-piperidin-1-yl]-(1-phenethyl-1H-pyrrolo[3,2-b]pyridin-3-yl)-methanone dihydrochloride

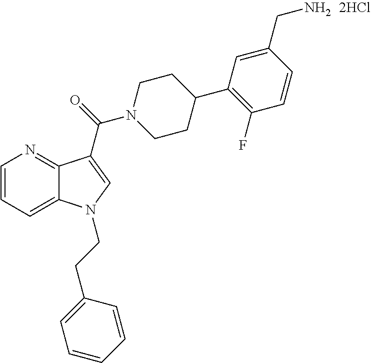

### A. 1-Phenethyl-1H-pyrrolo[3,2-b]pyridine

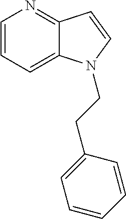

The title compound is prepared in a similar manner as Example 12C using 1H-pyrrolo[3,2-b]pyridine and phenethyl bromide as the starting materials. ¹H NMR (CDCl₃, 300 MHz) δ 8.25 (d, 1H), 7.50 (d, 1H), 7.50-6.90 (m, 7H), 6.65 (s, 1H), 4.35 (s, 2H), 3.10 (s, 3H). LC 1.62 min; MS m/z 223 (M+1, 100%).

### B 3-Iodo-1-phenethyl-1H-pyrrolo[3,2-b]pyridine

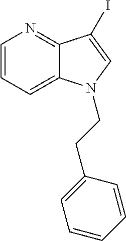

To a solution of 1-phenethyl-1H-pyrrolo[3,2-b]pyridine (400 mg, 1.8 mmol) in THF (6 mL) is added N-iodosuccimide (526 mg, 1.3 mmol). This reaction mixture is stirred at rt for 2 h and then at 45° C. for 2 h. The solvent is removed in vacuo, and the residue is dissolved in Et₂O, washed with 0.5 M NaOH (2×), H₂O, and brine, dried over Na₂SO₄, filtered, and concentrated in vacuo to yield 540 mg (86%) of the product as a yellow solid. ¹H NMR (CDCl₃, 300 MHz) δ 8.55 (s, 1H), 7.45 (d, 1H), 7.15-7.05 (m, 1H), 7.09-6.65 (m, 1H), 4.35 (s, 2H), 3.10 (s, 3H). LC 0.77 min; MS m/z 249 (M+1, 100%).

### C. 1-Phenethyl-1H-pyrrolo[3,2-b]pyridine-3-carboxylic acid ethyl ester

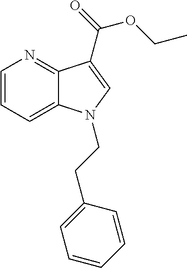

The title compound is prepared in a similar manner as Example 15I using 3-iodo-1-phenethyl-1H-pyrrolo[3,2-b]pyridine as the starting material. ¹H NMR (CDCl₃, 300 MHz) δ 8.65 (s, 1H), 7.85 (s, 1H), 7.50 (d, 1H), 7.40-6.80 (m, 6H), 4.60-4.25 (m, 4H), 3.20-2.95 (m, 2H), 1.40 (t, 3H). LC 0.65 min; MS m/z 295 (M+1, 100%).

### D. 1-Phenethyl-1H-pyrrolo[3,2-b]pyridine-3-carboxylic acid hydrochloride

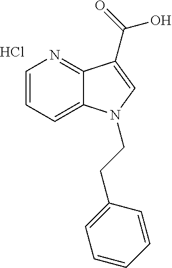

A mixture of 1-phenethyl-1H-pyrrolo[3,2-b]pyridine-3-carboxylic acid ethyl ester (290 mg, 0.98 mmol) in MeOH (2.9 mL) and NaOH (1 M, 2.9 mL) is stirred at rt for 2.5 h and then at 70° C. for 1 h. The pH of the reaction is adjusted to ˜2 with 1 M HCl. The resulting solution is concentrated to dryness in vacuo. The crude material is used in the next setp without further purification. LC 0.55 min; MS m/z 267 (M+1).

### E. 2,2,2-Trifluoro-N-{4-fluoro-3-[1-(1-phenethyl-1H-pyrrolo[3,2-b]pyridine-3-carbonyl)-piperidin-4-yl]-benzyl}-acetamide

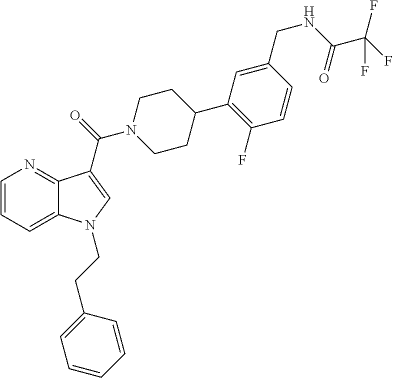

The title compound is prepared in a similar manner as Example 1C using 1-phenethyl-1H-pyrrolo[3,2-b]pyridine-3-carboxylic acid hydrochloride as the starting material. ¹H NMR (CDCl₃, 300 MHz) δ 8.60-8.50 (m, 1H), 7.62 (s, 1H), 7.60-7.50 (m, 1H), 7.40-6.90 (m, 9H), 6.65 (br s, 1H), 4.60 (br m, 1H), 4.50 (d, 2H), 4.38 (t, 2H), 3.40-2.85 (m, 6H), 2.00-1.70 (m, 4H). LC Rt 0.82 min; MS m/z 553 (M+1).

### F. [4-(5-Aminomethyl-2-fluoro-phenyl)-piperidin-1-yl]-(1-phenethyl-1H-pyrrolo[3,2-b]pyridin-3-yl)-methanone dihydrochloride

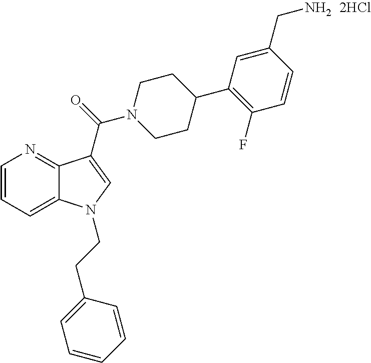

The title compound is prepared in a similar manner as Example 1D using 2,2,2-trifluoro-N-{4-fluoro-3-[1-(1-phenethyl-1H-pyrrolo[3,2-b]pyridine-3-carbonyl)-piperidin-4-yl]-benzyl}-acetamide as the starting material. ¹H NMR (DMSO-d6, 300 MHz) δ 8.79 (d, 1H), 8.61 (d, 1H), 8.55-8.30 (m, 4H), 7.75-7.55 (m, 2H), 7.45-7.30 (m, 1H), 7.30-6.95 (m, 7H), 4.80-4.65 (m, 2H), 4.55 (br m, 1H), 4.10-3.90 (m, 2H), 3.30-2.90 (m, 6H), 1.95-1.55 (m, 4H). LC 0.58 min; MS m/z 457 (M+1), 241.

### Example 17

### [4-(5-Aminomethyl-2-fluoro-phenyl)-piperidin-1-yl]-[1-(2-piperidin-1-yl-ethyl)-1H-pyrrolo[3,2-b]pyridin-3-yl]-methanone trihydrochloride

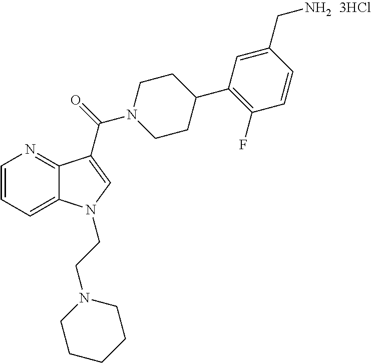

### A. 3-Iodo-1H-pyrrolo[3,2-b]pyridine

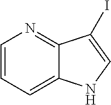

To a solution of 1H-pyrrolo[3,2-b]pyridine (1.94 g, 16.4 mmol) in THF (10 mL) is added N-iodosuccimide (4.06 g, 18.1 mmol). Precipitation occurs after a few minutes. The reaction is continued to stri at rt overnight. The precipitate is collected by filtration and is washed with a small amount of THF and heptane. The resulting white solid is dried in vacuo. The yield of the reaction is 4.1 g (quantitative). ¹H NMR (DMSO-d6, 300 MHz) δ 8.40 (s, 1H), 7.90-7.70 (m, 2H), 7.15 (d, 1H). LC 0.41 min; MS m/z 245 (M+1).

### B. 3-Iodo-1-(2-piperidin-1-yl-ethyl)-1H-pyrrolo[3,2-b]pyridine

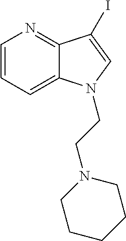

The title compound is prepared in a similar manner as Example 12C using 3-iodo-1H-pyrrolo[3,2-b]pyridine and N-(2-chloroethyl)piperidine hydrochloride as the starting materials. ¹H NMR (CDCl₃, 300 MHz) δ 8.60 (s, 1H), 7.65 (d, 1H), 7.55 (s, 1H), 7.15 (d, 1H), 4.20 (t, 2H), 3.65 (t, 2H), 2.50-2.30 (m, 4H), 1.70-1.35 (m, 6H). LC 0.43 min; MS m/z 356 (M+1).

### C. 1-(2-Piperidin-1-yl-ethyl)-1H-pyrrolo[3,2-b]pyridine-3-carboxylic acid ethyl ester

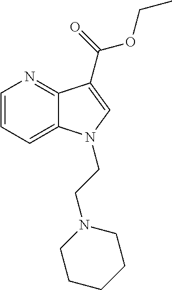

The title compound is prepared in a similar manner as Example 15I using 3-iodo-1-(2-piperidin-1-yl-ethyl)-1H-pyrrolo[3,2-b]pyridine as the starting material. ¹H NMR (CDCl₃, 300 MHz) δ 8.65 (d, 1H), 8.10 (s, 1H), 7.70 (d, 1H), 7.20-7.10 (m, 1H), 4.45 (q, 2H), 4.20 (t, 2H), 3.70 (t, 2H), 2.50-2.30 (m, 4H), 1.65-1.35 (m, 9H). LC 0.41 min; MS m/z 302 (M+1).

### D. 1-(2-Piperidin-1-yl-ethyl)-1H-pyrrolo[3,2-b]pyridine-3-carboxylic acid dihydrochloride

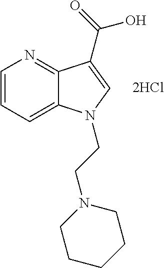

A mixture of 1-(2-piperidin-1-yl-ethyl)-1H-pyrrolo[3,2-b]pyridine-3-carboxylic acid ethyl ester (110 mg, 0.36 mmol) in MeOH (1.1 mL) and NaOH (1 M, 1.1 mL) is stirred at rt for 2.5 h and then at 70° C. for 1 h. The reaction mixture is concentrated in vacuo to remove the methanol. The pH of the resulting mixture is adjusted to ˜4 with 3 M HCl. The resulting solution is concentrated to dryness in vacuo. The crude material is used in the next step without further purification.

### E. 2,2,2-Trifluoro-N-(4-fluoro-3-{1-[1-(2-piperidin-1-yl-ethyl)-1H-pyrrolo[3,2-b]pyridine-3-carbonyl]-piperidin-4-yl}-benzyl)-acetamide

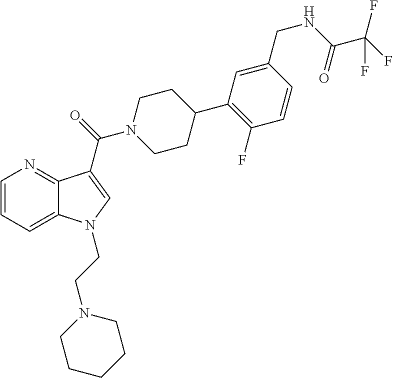

The title compound is prepared in a similar manner as Example 1C using 1-(2-piperidin-1-yl-ethyl)-1H-pyrrolo[3,2-b]pyridine-3-carboxylic acid dihydrochloride as the starting material. ¹H NMR (CDCl₃, 300 MHz) δ 8.60-8.50 (m, 1H), 7.62 (s, 1H), 7.75-7.65 (m, 1H), 7.25-6.95 (m, 4H), 6.60 (br s, 1H), 4.70 (br m, 1H), 4.59 (d, 2H), 4.24 (t, 2H), 3.35-2.85 (m, 4H), 2.73 (t, 2H), 2.55-2.35 (m, 4H), 2.00-1.75 (m, 4H), 1.70-1.35 (m, 6H). LC Rt 0.63 min; MS m/z 560 (M+1), 281.

### F. [4-(5-Aminomethyl-2-fluoro-phenyl)-piperidin-1-yl]-[1-(2-piperidin-1-yl-ethyl)-1H-pyrrolo[3,2-b]pyridin-3-yl]-methanone trihydrochloride

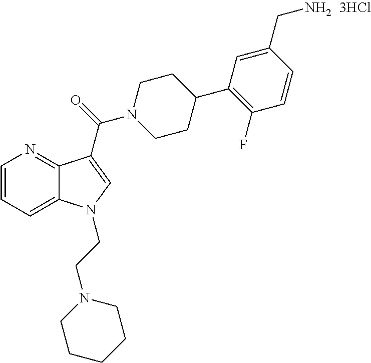

To a mixture of 2,2,2-trifluoro-N-(4-fluoro-3-{1-[1-(2-piperidin-1-yl-ethyl)-1H-pyrrolo[3,2-b]pyridine-3-carbonyl]-piperidin-4-yl}-benzyl)-acetamide (85 mg, 0.15 mmol) in MeOH (5 mL) is added aqueous K₂CO₃ (168 mg, 1.21 mmol, dissolved in 1 mL H₂O). This mixture is stirred at rt 1 h and then 45° C. for 1.5 h. LC/MS indicates the reaction is completed. The reaction mixture is concentrated in vacuo to remove most of the methanol, and the residue is dissolved in H₂O. The solution is acidified to pH 3 with 3 M HCl. The resulting solution is filtered, and the filtrate is purified by HPLC to give 42 mg (48%) of the product as a white solid. ¹H NMR (DMSO-d6, 300 MHz) δ 10.8 (br s, 1H), 8.51 (d, 1H), 8.45-8.10 (m, 6H), 7.60-7.45 (m, 1H), 7.45-7.30 (m, 2H), 7.30-7.15 (m, 1H), 4.90-4.80 (m, 2H), 4.70 (br m, 1H), 4.10-3.90 (m, 2H), 3.25-2.75 (m, 6H), 2.50-2.30 (m, 4H), 1.90-1.50 (m, 8H), 1.50-1.25 (m, 2H). LC 0.40 min; MS m/z 464 (M+1), 192.

### Example 18

### [4-(5-Aminomethyl-2-fluoro-phenyl)-piperidin-1-yl]-[1-(2-pyrrolidin-1-yl-ethyl)-1H-pyrrolo[3,2-b]pyridin-3-yl]-methanone trihydrochloride

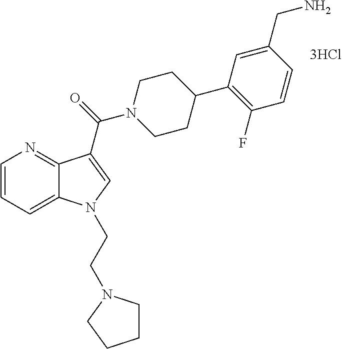

### A. 3-Iodo-1-(2-pyrrolidin-1-yl-ethyl)-1H-pyrrolo[3,2-b]pyridine

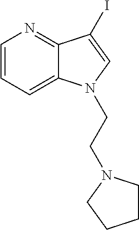

The title compound is prepared in a similar manner as Example 12C using 3-iodo-1H-pyrrolo[3,2-b]pyridine and N-(2-chloroethyl)piperidine hydrochloride as the starting materials. ¹H NMR (CDCl₃, 300 MHz) δ 8.65 (s, 1H), 7.65 (d, 1H), 7.55 (s, 1H), 7.15 (d, 1H), 4.25 (t, 2H), 2.80 (t, 2H), 2.70-2.50 (m, 4H), 1.90-1.70 (m, 4H). LC 0.54 min; MS m/z 342 (M+1), 192.

### B. 1-(2-Pyrrolidin-1-yl-ethyl)-1H-pyrrolo[3,2-b]pyridine-3-carboxylic acid ethyl ester

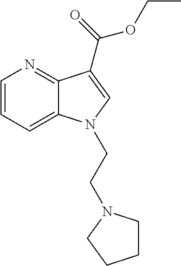

The title compound is prepared in a similar manner as Example 15I using 3-iodo-1-(2-pyrrolidin-1-yl-ethyl)-1H-pyrrolo[3,2-b]pyridine as the starting material. ¹H NMR (CDCl₃, 300 MHz) δ 8.70 (d, 1H), 8.10 (s, 1H), 7.75 (d, 1H), 7.25-7.10 (m, 1H), 4.45 (q, 2H), 4.25 (t, 2H), 2.90 (t, 2H), 2.60-2.40 (m, 4H), 1.85-1.70 (m, 4H), 1.45 (t, 3H). LC 0.55 min; MS m/z 288 (M+1), 165.

### C. 1-(2-Pyrrolidin-1-yl-ethyl)-1H-pyrrolo[3,2-b]pyridine-3-carboxylic acid dihydrochloride

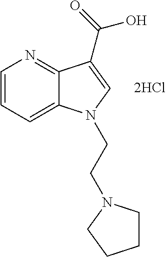

A mixture of 1-(2-pyrrolidin-1-yl-ethyl)-1H-pyrrolo[3,2-b]pyridine-3-carboxylic acid ethyl ester (105 mg, 0.44 mmol) in MeOH (1.1 mL) and NaOH (1 M, 1.1 mL) is stirred at at 70° C. for 1 h. The reaction mixture is concentrated in vacuo to remove the methanol. The pH of the resulting mixture is adjusted to ˜4 with 3M HCl. The resulting solution is concentrated to dryness in vacuo. The crude material is used in the next step without further purification.

### D. 2,2,2-Trifluoro-N-(4-fluoro-3-{1-[1-(2-pyrrolidin-1-yl-ethyl)-1H-pyrrolo[3,2-b]pyridine-3-carbonyl]-piperidin-4-yl}-benzyl)-acetamide

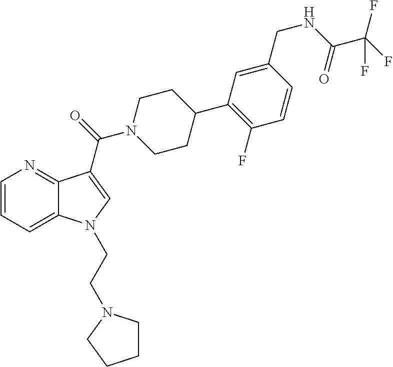

The title compound is prepared in a similar manner as Example 1C using 1-(2-pyrrolidin-1-yl-ethyl)-1H-pyrrolo[3,2-b]pyridine-3-carboxylic acid dihydrochloride as the starting material. ¹H NMR (CDCl₃, 300 MHz) δ 8.60-8.50 (m, 1H), 7.88 (s, 1H), 7.75-7.65 (m, 1H), 7.25-6.95 (m, 4H), 6.70 (br m, 1H), 4.75 (br m, 1H), 4.49 (d, 2H), 4.28 (t, 2H), 3.35-3.30 (m, 4H), 2.93 (t, 2H), 2.50-2.30 (m, 4H), 2.05-1.65 (m, 8H). LC Rt 0.62 min; MS m/z 546 (M+1), 217.

### E. [4-(5-Aminomethyl-2-fluoro-phenyl)-piperidin-1-yl]-[1-(2-pyrrolidin-1-yl-ethyl)-1H-pyrrolo[3,2-b]pyridin-3-yl]-methanone trihydrochloride

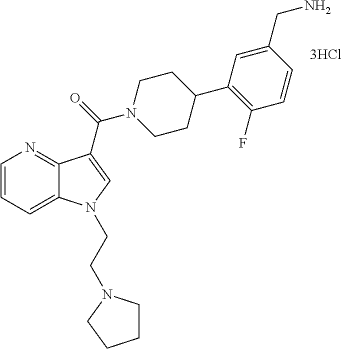

To a mixture of 2,2,2-trifluoro-N-(4-fluoro-3-{1-[1-(2-pyrrolidin-1-yl-ethyl)-1H-pyrrolo[3,2-b]pyridine-3-carbonyl]-piperidin-4-yl}-benzyl)-acetamide (110 mg, 0.20 mmol) in MeOH (5 mL) is added aqueous K₂CO₃ (61 mg, 1.61 mmol, dissolved in 1 mL H₂O). This mixture is stirred at rt 1 h and then 45° C. for 2 h. LC/MS indicates the reaction is completed. The reaction mixture is concentrated in vacuo to remove most of the methanol, and the residue is dissolved in H₂O. The solution is acidified to pH 3 with 3 M HCl. The resulting solution is filtered, and the filtrate is purified by HPLC to give 112 mg (quantitative) of the product as a light yellow solid. ¹H NMR (DMSO-d6, 300 MHz) δ 10.2 (br s, 1H), 8.55 (d, 1H), 8.41 (d, 1H), 8.33 9s, 1H), 8.19 (br s, 4H), 7.60-7.40 (m, 2H), 7.40-7.30 (m, 1H), 7.10-6.95 (m, 1H), 4.80-4.60 (m, 2H), 4.30 (br m, 1H), 4.10-3.95 (m, 2H), 3.90-3.65 (m, 2H), 3.65-3.45 (m, 2H), 3.20-2.80 (m, 4H), 2.15-1.50 (m, 10H). LC 0.378 min; MS m/z 450 (M+1).

### Example 19

### [4-(5-Aminomethyl-2-fluoro-phenyl)-piperidin-1-yl]-(1H-pyrrolo[2,3-b]pyridin-3-yl)-methanone dihydrochloride

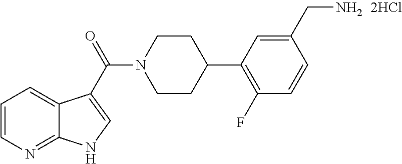

### A. 2,2,2-Trifluoro-N-{4-fluoro-3-[1-(1H-pyrrolo[2,3-b]pyridine-3-carbonyl)-piperidin-4-yl]-benzyl}-acetamide

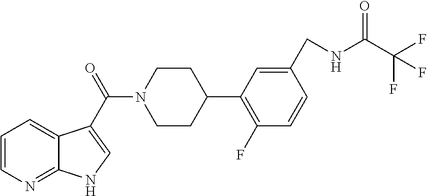

The title compound is prepared in a similar manner as Example 1C using 1H-pyrrolo[2,3-b]pyridine-3-carboxylic acid as the starting material. ¹H-NMR (CDCl₃, 300 MHz) δ 9.8 (bs, 1H), 8.4 (m, 1H), 8.2 (m, 1H), 7.6 (d, 1H), 7.3-7.0 (m, 3H), 6.75 (bs, 1H), 4.6 (m, 2H), 4.45 (m, 2H), 3.2 (m, 3H), 2.5 (m, 1H), 1.9 (m, 2H), 1.8 (m, 2H). LCMS m/z 449 (M+H).

### B. [4-(5-Aminomethyl-2-fluoro-phenyl)-piperidin-1-yl]-(1H-pyrrolo[2,3-b]pyridin-3-yl)-methanone dihydrochloride

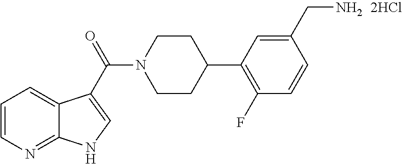

The title compound is prepared in a similar manner as Example 5D using 2,2,2-trifluoro-N-{4-fluoro-3-[1-(1H-pyrrolo[2,3-b]pyridine-3-carbonyl)-piperidin-4-yl]-benzyl}-acetamide as the starting material. ¹H NMR (DMSO-d6, 300 MHz) δ 12.4 (bs, 1H), 8.4 (bs, 2H), 8.2 (m, 1H), 7.9 (m, 1H), 7.6 (m, 1H), 7.4 (m, 1H), 7.2 (m, 2H), 4.4 (m, 2H), 4.0 (m, 2H), 3.2 (m, 3H), 1.95-1.8 (m, 4H). LCMS m/z 353 (M+H).

### Example 20

### [4-(5-Aminomethyl-2-fluoro-phenyl)-piperidin-1-yl]-[1-(2-methoxy-ethyl)-1H-pyrrolo[2,3-b]pyridin-3-yl]-methanone dihydrochloride

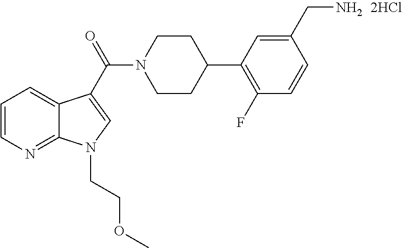

### A. 1H-Pyrrolo[2,3-b]pyridine-3-carboxylic acid methyl ester

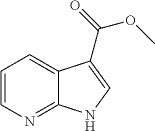

The title compound is prepared in a similar manner as Example 2A using 1H-pyrrolo[2,3-b]pyridine-3-carboxylic acid as the starting material. ¹H-NMR (CDCl₃, 300 MHz) δ 10.5 (bs, 1H), 8.5 (m, 1H), 8.4 (m, 1H), 8.1 (m, 1H), 7.2 (m, 1H), 3.95 (s, 3H). LCMS m/z 177 (M+H).

### B. 1-(2-Methoxy-ethyl)-1H-pyrrolo[2,3-b]pyridine-3-carboxylic acid methyl ester

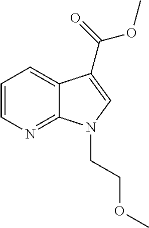

The title compound is prepared in a similar manner as Example 2B using 1H-pyrrolo[2,3-b]pyridine-3-carboxylic acid methyl ester as the starting material. ¹H-NMR (CDCl₃, 300 MHz) δ 8.4 (m, 2H), 8.1 (s, 1H), 7.2 (m, 1H), 4.5 (t, 2H), 3.9 (s, 3H), 3.8 (t, 2H), 3.3 (s, 3H). LCMS m/z 235 (M+H).

### C. 1-(2-Methoxy-ethyl)-1H-pyrrolo[2,3-b]pyridine-3-carboxylic acid

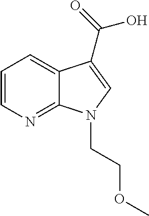

The title compound is prepared in a similar manner as Example 8C using 1-(2-methoxy-ethyl)-1H-pyrrolo[2,3-b]pyridine-3-carboxylic acid methyl ester as the starting material. ¹H-NMR (DMSO-d6, 300 MHz) δ 8.3 (m, 2H), 8.2 (s, 1H), 7.3 (m, 1H), 4.5 (t, 2H), 3.75 (t, 2H), 3.2 (s, 3H). LCMS m/z 221 (M+H).

### D. 2,2,2-Trifluoro-N-(4-fluoro-3-{1-[1-(2-methoxy-ethyl)-1H-pyrrolo[2,3-b]pyridine-3-carbonyl]-piperidin-4-yl}-benzyl)-acetamide

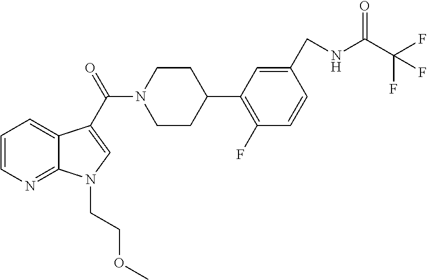

The title compound is prepared in a similar manner as Example 1C using 1-(2-methoxy-ethyl)-1H-pyrrolo[2,3-b]pyridine-3-carboxylic acid as the starting material. ¹H-NMR (CDCl₃, 300 MHz) δ 8.35 (m, 1H), 8.1 (m, 1H), 7.7 (s, H), 7.2 (m, 3H), 7.1 (m, 1H), 6.6 (bs, 1H), 4.65 (m, 2H), 4.5 (m, 4H), 3.8 (m, 2H), 3.3 (s, 3H), 3.15 (m, 3H), 1.9 (m, 2H), 1.8 (m, 2H). LCMS m/z 507 (M+H).

### E. [4-(5-Aminomethyl-2-fluoro-phenyl)-piperidin-1-yl]-[1-(2-methoxy-ethyl)-1H-pyrrolo[2,3-b]pyridin-3-yl]-methanone hydrochloride

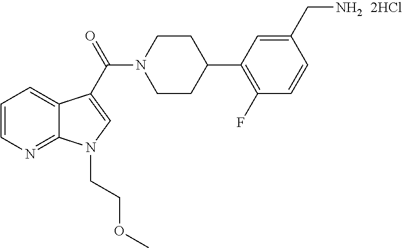

The title compound is prepared in a similar manner as Example 5D using 2,2,2-trifluoro-N-(4-fluoro-3-{1-[1-(2-methoxy-ethyl)-1H-pyrrolo[2,3-b]pyridine-3-carbonyl]-piperidin-4-yl}-benzyl)-acetamide as the starting material. ¹H NMR (DMSO-d6, 300 MHz) δ 8.4 (m, 1H), 8.2 (bs, 2H), 8.1 (m, 1H), 7.9 (s, 1H), 7.6 (m, 1H), 7.4 (m, 1H), 7.2 (m, 2H), 4.5 (m, 4H), 4.0 (m, 2H), 3.7 (m, 2H), 3.25 (s, 3H), 3.2 (m, 3H), 1.9 (m, 2H), 1.75 (m, 2H). LCMS m/z 411 (M+H).

### Biological Activity

The properties of the compound of the present invention are demonstrated by: 1) its beta-Tryptase Inhibitory Potency (IC₅₀ and Kᵢ values).

### In Vitro Test Procedure

As all the actions of tryptase, as described in the background section, are dependent on its catalytic activity, then compounds that inhibit its catalytic activity will potentially inhibit the actions of tryptase. Inhibition of this catalytic activity may be measured by the in vitro enzyme assay and the cellular assay.

Tryptase inhibition activity is confirmed using either isolated human lung tryptase or recombinant human beta tryptase expressed in yeast cells. Essentially equivalent results are obtained using isolated native enzyme or the expressed enzyme. The assay procedure employs a 96 well microplate (Costar 3590) using L-pyroglutamyl-L-prolyl-L-arginine-para-nitroanilide (S2366: Quadratech) as substrate (essentially as described by McEuen et. al. Biochem Pharm, 1996, 52, pages 331-340). Assays are performed at room temperature using 0.5 mM substrate (2×Kₘ) and the microplate is read on a microplate reader (Beckman Biomek Plate reader) at 405 nm wavelength.

### Materials and Methods for Tryptase Primary Screen (Chromogenic Assay)

### Assay Buffer

50 mM Tris (pH 8.2), 100 mM NaCl, 0.05% Tween 20, 50 μg/mL heparin.

### Substrate

S2366 (Stock solutions of 2.5 mM).

### Enzyme

Purified recombinant beta Tryptase Stocks of 310 μg/mL.

### Protocol (Single Point Determination)

Add 60 μL of diluted substrate (final concentration of 500 μM in assay buffer) to each well
Add compound in duplicates, final concentration of 20 μM, volume 20 μL
Add enzyme at a final concentration of 50 ng/mL in a volume of 20 μL
Total volume for each well is 100 μL
Agitate briefly to mix and incubate at room temp in the dark for 30 minutes
Read absorbencies at 405 nM
   Each plate has the following controls:
Totals: 60 μL of substrate, 20 μL of buffer (with 0.2% final concentration of DMSO), 20 μL of enzyme
Non-specific: 60 μL of substrate, 40 μL of buffer (with 0.2% DMSO)
Totals: 60 μL of substrate, 20 μL of buffer (No DMSO), 20 μL of enzyme
Non-specific: 60 μL of substrate, 40 μL of buffer (No DMSO)

### Protocol (IC₅₀ and K Determination)

The protocol is essentially the same as above except that the compound is added in duplicates at the following final concentrations: 0.01, 0.03, 0.1, 0.3, 1, 3, 10 μM (All dilutions carried out manually). For every assay, whether single point or IC₅₀ determination, a standard compound is used to derive IC₅₀ for comparison. From the IC₅₀ value, the Kᵢ can be calculated using the following formula: Kᵢ=IC₅₀/(1+[Substrate]/Kₘ).

The beta-Tryptase inhibitory potency for the compound of formula I is Kᵢ value of 26 nM.

TABLE 1
Activity of compounds against beta-Tryptase

| EXAMPLE | Tryptase Ki (nM) |
|---|---|
| 1 | 221 |
| 2 | 538 |
| 3 | 915 |
| 4 | 1100 |
| 5 | 220 |
| 6 | 51% @ 10 mM |
| 7 | 37% @ 10 mM |
| 8 | 99 |
| 9 | 35 |
| 10 | 180 |
| 11 | 166 |
| 12 | 23, 13 |
| 13 | 25 |
| 14 | 25 |
| 15 | 9.9 |
| 16 | 2.9 |
| 17 | 290 |
| 18 | 277 |
| 19 | 203 |
| 20 | 92 |

We claim:

1. A compound selected from the group consisting of:
[4-(5-aminomethyl-2-fluoro-phenyl)-piperidin-1-yl]-[1-(2-methoxy-ethyl)-7-methyl-1H-pyrrolo[3,2-b]pyridin-3-yl]-methanone,
[4-(5-aminomethyl-2-fluoro-phenyl)-piperidin-1-yl]-[7-chloro-1-(2-methoxy-ethyl)-1H-pyrrolo[2,3-c]pyridin-3-yl]-methanone,
[4-(5-aminomethyl-2-fluoro-phenyl)-piperidin-1-yl]-[7-methyl-1-(2-methoxy-ethyl)-1H-pyrrolo[2,3-c]pyridin-3-yl]-methanone,
[4-(5-aminomethyl-2-fluoro-phenyl)-piperidin-1-yl]-(1-phenethyl-1H-pyrrolo[3,2-b]pyridin-3-yl)-methanone,
[4-(5-aminomethyl-2-fluoro-phenyl)-piperidin-1-yl]-[1-(2-methoxy-ethyl)-1H-pyrrolo[2,3-c]pyridin-3-yl]-methanone,
[4-(5-aminomethyl-2-fluoro-phenyl)-piperidin-1-yl]-[1-(2-methoxy-ethyl)-1H-pyrrolo[2,3-b]pyridin-3-yl]-methanone,
[4-(3-aminomethyl-phenyl)-piperidin-1-yl]-[1-(2-methoxy-ethyl)-1H-pyrrolo[2,3-c]pyridin-3-yl]-methanone,
[4-(5-aminomethyl-2-fluoro-phenyl)-piperidin-1-yl]-[7-(2-methoxy-ethoxy)-1-(2-methoxy-ethyl)-1H-pyrrolo[2,3-c]pyridin-3-yl]-methanone,
[4-(5-aminomethyl-2-fluoro-phenyl)-piperidin-1-yl]-[7-methoxy-1-(2-methoxy-ethyl)-1H-pyrrolo[3,2-b]pyridin-3-yl]-methanone,
[4-(5-aminomethyl-2-fluoro-phenyl)-piperidin-1-yl]-[1-(2-methoxy-ethyl)-1H-pyrrolo[3,2-c]pyridin-3-yl]-methanone,
[4-(5-aminomethyl-2-fluoro-phenyl)-piperidin-1-yl]-(1H-pyrrolo[2,3-b]pyridin-3-yl)-methanone,
[4-(5-aminomethyl-2-fluoro-phenyl)-piperidin-1-yl]-(1H-pyrrolo[2,3-c]pyridin-3-yl)-methanone,
[4-(5-aminomethyl-2-fluoro-phenyl)-piperidin-1-yl]-(1H-pyrrolo[2,3-b]pyridin-2-yl)-methanone,
[4-(5-aminomethyl-2-fluoro-phenyl)-piperidin-1-yl]-[1-(2-pyrrolidin-1-yl-ethyl)-1H-pyrrolo[3,2-b]pyridin-3-yl]-methanone,
[4-(5-aminomethyl-2-fluoro-phenyl)-piperidin-1-yl]-[1-(2-piperidin-1-yl-ethyl)-1H-pyrrolo[3,2-b]pyridin-3-yl]-methanone,
[4-(5-aminomethyl-2-fluoro-phenyl)-piperidin-1-yl]-[1-(2-methoxy-ethyl)-1H-pyrrolo[2,3-b]pyridin-2-yl]-methanone,
[4-(3-aminomethyl-phenyl)-piperidin-1-yl]-[1-(2-methoxy-ethyl)-1H-pyrrolo[2,3-b]pyridin-2-yl]-methanone,
[4-(5-aminomethyl-2-fluoro-phenyl)-piperidin-1-yl]-[1-(2-methoxy-ethyl)-1H-pyrrolo[3,2-c]pyridin-2-yl]-methanone,
[4-(3-aminomethyl-phenyl)-piperidin-1-yl]-[1-(2-methoxy-ethyl)-1H-pyrrolo[3,2-b]pyridin-2-yl]-methanone, and
[4-(5-aminomethyl-2-fluoro-phenyl)-piperidin-1-yl]-[1-(2-methoxy-ethyl)-1H-pyrrolo[3,2-b]pyridin-2-yl]-methanone.

2. A compound as recited in claim 1 that is selected from the group consisting of:
[4-(3-Aminomethyl-phenyl)-piperidin-1-yl]-[1-(2-methoxy-ethyl)-1H-pyrrolo[2,3-c]pyridin-3-yl]-methanone,
[4-(5-Aminomethyl-2-fluoro-phenyl)-piperidin-1-yl]-(1H-pyrrolo[2,3-b]pyridin-3-yl)-methanone,
[4-(5-Aminomethyl-2-fluoro-phenyl)-piperidin-1-yl]-(1-phenethyl-1H-pyrrolo[3,2-b]pyridin-3-yl)-methanone,
[4-(5-Aminomethyl-2-fluoro-phenyl)-piperidin-1-yl]-[1-(2-methoxy-ethyl)-1H-pyrrolo[2,3-b]pyridin-3-yl]-methanone,
[4-(5-Aminomethyl-2-fluoro-phenyl)-piperidin-1-yl]-[1-(2-methoxy-ethyl)-1H-pyrrolo[2,3-c]pyridin-3-yl]-methanone,
[4-(5-Aminomethyl-2-fluoro-phenyl)-piperidin-1-yl]-[1-(2-methoxy-ethyl)-1H-pyrrolo[3,2-c]pyridin-3-yl]-methanone,
[4-(5-Aminomethyl-2-fluoro-phenyl)-piperidin-1-yl]-[1-(2-methoxy-ethyl)-7-methyl-1H-pyrrolo[3,2-b]pyridin-3-yl]-methanone,
[4-(5-Aminomethyl-2-fluoro-phenyl)-piperidin-1-yl]-[7-(2-methoxy-ethoxy)-1-(2-methoxy-ethyl)-1H-pyrrolo[2,3-c]pyridin-3-yl]-methanone,
[4-(5-Aminomethyl-2-fluoro-phenyl)-piperidin-1-yl]-[7-chloro-1-(2-methoxy-ethyl)-1H-pyrrolo[2,3-c]pyridin-3-yl]-methanone,
[4-(5-Aminomethyl-2-fluoro-phenyl)-piperidin-1-yl]-[7-methoxy-1-(2-methoxy-ethyl)-1H-pyrrolo[3,2-b]pyridin-3-yl]-methanone, and
[4-(5-Aminomethyl-2-fluoro-phenyl)-piperidin-1-yl]-[7-methyl-1-(2-methoxy-ethyl)-1H-pyrrolo[2,3-c]pyridin-3-yl]-methanone.

3. A compound as recited in claim 1 that is selected from the group consisting of:
[4-(5-Aminomethyl-2-fluoro-phenyl)-piperidin-1-yl]-(1-phenethyl-1H-pyrrolo[3,2-b]pyridin-3-yl)-methanone;
[4-(5-Aminomethyl-2-fluoro-phenyl)-piperidin-1-yl]-[1-(2-methoxy-ethyl)-1H-pyrrolo[2,3-c]pyridin-3-yl]-methanone;
[4-(5-Aminomethyl-2-fluoro-phenyl)-piperidin-1-yl]-[1-(2-methoxy-ethyl)-7-methyl-1H-pyrrolo[3,2-b]pyridin-3-yl]-methanone;
[4-(5-Aminomethyl-2-fluoro-phenyl)-piperidin-1-yl]-[7-chloro-1-(2-methoxy-ethyl)-1H-pyrrolo[2,3-c]pyridin-3-yl]-methanone;
[4-(5-Aminomethyl-2-fluoro-phenyl)-piperidin-1-yl]-[7-methoxy-1-(2-methoxy-ethyl)-1H-pyrrolo[3,2-b]pyridin-3-yl]-methanone; and
[4-(5-Aminomethyl-2-fluoro-phenyl)-piperidin-1-yl]-[7-methyl-1-(2-methoxy-ethyl)-1H-pyrrolo[2,3-c]pyridin-3-yl]-methanone.

4. A pharmaceutical composition comprising a compound as recited in claim 1 in a pharmaceutically acceptable carrier.

5. A method for the treatment of an inflammatory disease comprising the administration of a pharmaceutically effective amount of a compound as recited in claim 1.

6. A method for the treatment of an inflammatory disease comprising the administration of a pharmaceutically effective amount of a composition as recited in claim 4.
